(19) United States
(12) Patent Application Publication
(10) Pub. No.: US 2021/0251934 A1
(43) Pub. Date: Aug. 19, 2021

(54) NOVEL COMPOSITIONS FOR THE TREATMENT OF INFLAMMATORY DISEASES

(71) Applicant: SystaMedic Inc., Stonington, CT (US)

(72) Inventors: Anton F. Fliri, Stonington, CT (US); Shama Kajiji, Mystic, CT (US); Palaniyandi Manivasakam, West Roxbury, MA (US)

(73) Assignee: SystaMedic Inc., Stonington, CT (US)

(21) Appl. No.: 17/231,674

(22) Filed: Apr. 15, 2021

Related U.S. Application Data
(63) Continuation-in-part of application No. PCT/US2019/056238, filed on Oct. 15, 2019.
(60) Provisional application No. 62/746,098, filed on Oct. 16, 2018.

(51) Int. Cl. A61K 31/202; A61K 31/047; A61K 35/60; A61K 31/09; A61K 31/167; A61K 31/522; A61K 31/352; A61K 45/06; A61K 47/06; A61K 31/155

(52) U.S. Cl. CPC A61K 31/202; A61K 31/047; A61K 35/60; A61K 31/09; A61K 31/155; A61K 31/522; A61K 31/352; A61K 45/06; A61K 47/06; A61K 31/167

(57) ABSTRACT

Anti-inflammatory compositions that merely treat symptoms of inflammation and not its root causes have frequently been shown to impair healing processes and exacerbate disease. The present disclosure includes novel compositions, combinations and methods of treatment that overcome this shortcoming by down regulating excessive inflammation and by activating cellular functions supporting healing processes. These novel compositions include combinations of ingredients selected from three groups wherein the first group of ingredients includes effective amounts of herring roe proteins, krill proteins, herring roe oil, krill oil, fish oils, docosahexaenoic acid, docosapentaenoic acid, eicosapentaenoic acid, stearidonic acid, alpha linoleic acid, gamma linoleic acid, dihomo gamma-linolenic acid, the lithium and zinc salts of said fatty acids; the second group of ingredients includes effective amounts of phosphatidyl inositol, inositol, aspirin; and the third group of ingredients includes effective amounts of compositions capable of activating autophagy.

### CROSS-REFERENCE TO RELATED APPLICATIONS

This application is a continuation-in-part of International Application Number PCT/US2019/056238 filed Oct. 15, 2019, which claims priority to and the benefit of U.S. Provisional Patent Application Ser. No. 62/746,098 filed Oct. 16, 2018, the disclosures of which are incorporated herein by reference in their entirety.

### FIELD

This disclosure relates to pharmaceutical compositions, pharmaceutical combinations and methods of treatment of inflammatory diseases.

### BACKGROUND

Inflammation, which is an essential part of the healing process, consists of multiple stages. Thus, in the first stage of inflammation, often called irritation, inflammatory mediators are produced and this stage transitions into inflammation. This stage results in production and discharge of pus and the final stages involve activation of wound healing processes. Without inflammation, infections and wounds would never heal. Thus, agents reducing inflammation have frequently been shown to impair healing processes (See, Giannoudis P. V. et al. 2000; Hauser R. A et al. 2010). For correcting these problems, pharmaceutical compositions are needed that do not only ameliorate symptoms of inflammation but also support healing processes. Among these healing processes is a process termed autophagy (An Y. et al. 2018). Thus, it has been shown that autophagy has not only a fundamental role in immunity but also limits inflammatory pathologies, contributes to the differentiation of myeloid and lymphoid cell types and facilitates memory responses. Likewise, autophagy is involved in re-epithelialization, and tissue remodeling (See, Li, M. et al. 2017), muscle repair (See, Fan J.et al. 2017), nerve regeneration and scare formation (See, Jessen K. R. et al. 2016). These functions establish an intimate link between autophagy, immunity, chronic inflammatory diseases and wound healing. Hence pharmacological compositions capable of down regulating inflammatory responses and selectively modulating autophagy associated with inflammatory disease are expected to improve current therapies of many diseases associated with chronic inflammation (See, Matsuzawa-Ishimoto, Y. et al. 2018).

One of the most common immune-mediated chronic, inflammatory diseases is Psoriasis which is characterized by epidermal hyperplasia, abnormal terminal differentiation of keratinocytes, infiltration of T cells, dendritic cells, macrophages and neutrophils into the dermis and epidermis, hyperproliferative keratinocytes, excessive angiogenesis and vasodilation (See, Raychaudhuri, 2013) and a thick cornified layer with retained nuclei or parakeratosis (See, Donetti et al. 2012; Kim et al. 2011; Wolberink et al. 2011) which is indicative of incomplete terminal differentiation. This disease has a genetic predisposition and its onset, triggered by environmental factors, predisposes affected individuals to metabolic disorders and life shortening. Thus, epidemiological and clinical studies have consistently shown that psoriasis is associated with increased cardiovascular risks (See, Armstrong et al. 2013), increased atherosclerosis (See, Maria Joao Paiva-Lopes et al. 2017), increased hyperlipidemia characterized by lower HDL concentration, decreases in LDL size (See, Nehal N.et al. 2012), increases in Lipoprotein A (LPA) levels (See, Latha K. P.2009), decreases in apo A-I levels (See, Seishima M. et al. 1994), decreases in paraoxonase-1 levels (See, Ferretti G. et al. 2012) and dysfunctional HDL particles (See, Holzer M. et al. 2013). For example, HDL isolated from psoriatic patients shows a significantly impaired capability to mobilize cholesterol from macrophages. In turn, successful anti-psoriatic therapy significantly improves HDL composition and functions independently of serum HDL-cholesterol levels (See, Marsche G. et al. 2014). In addition, a large group of genes with altered expression levels in psoriasis are related to fatty acid signaling and adipocyte differentiation and exhibit a pattern consistent with the activation of peroxisome proliferator-activated receptor delta (PPARδ) (See, Romanowska, M. et al. 2008). All of these factors implicate processes involved in regulating lipid homeostasis in psoriasis. Supporting this premise is the observation that single arm non-placebo controlled clinical studies with Cholesterol lowering drugs Statins show an improvement in Psoriasis associated symptom index (PASI) scores; however, analysis of placebo-controlled studies revealed that the efficacy of statins in therapy of psoriasis may be marginable and would need improvement in order to exert therapeutic impact (See, Ramessur R. et al. 2017).

Aside skin inflammation, psoriasis is frequently also associated with loss of bone mass, depressive disorders (See, Randa H. et al. 2017) and imbalances at various system levels including shifts in T cell subpopulations (See, Mavropoulos et al. 2017), alterations in skin microbial communities (See, Alekseyenko, A. V. et al. 2013), increased expression of the innate immune receptor TLR2 (See, Begon, E. et al. 2007), decreases in anti-inflammatory cytokines such as IL-10 (See, Mavropoulos 2017), imbalance between locally produced proresolution (resolvin, protectins and marensins) and proinflammatory modulators (See, Sorokin A.V. et al. 2018); increases in acute-phase protein serum amyloid A (SAA) which regulates inflammasome activation (See, Yu N et al. 2015); changes in histone deacetylases levels and overexpression of HDAC1 (Zhang P. et al. 2011), excess of pro-inflammatory cytokines such as IL 17 and IL-23 (See, Li J et al. 2007; Soderstrom C. et al. 2017); Increased expression of Nuclear factor kappa B and cyclo-oxygenase-2 COX2 (See, Bakry O. A. et al. 2015); elevated mRNA levels of the angiogenic chemokine stromal cell-derived factor-1 (SDF-1) and its receptor CXCR4 (See, Zgraggen S. et al. (2014); increased levels of branched chain amino acids such as leucine (See, Yan J. 2017); increased mitochondrial respiration coupled to ATP synthesis and resistance of keratinocyte mitochondria to pro-apoptotic stimuli (See, Rosella et al. 2014); decreased expression of superoxide dismutase (SOD), catalase (CAT) and paraxonase 1 (PON1) (See, Nemati H. 2014) ; decreased expression of the autophagy associated protein LC3-II (See, Varshney P. et al.2018), increased expression of the autophagy adaptor protein p62/SQSTM1 accompanied by defect autophagy and increased autophagy flux (See, Jin-Man Kim et al. 2010); increased expression of phosphatidylinositol 3-kinase (PIK3CA) (See, Zhang X et al. 1999); increased expression of the transcription factor STAT3 (See, Andres R. M. et al. 2013); decreased expression of transcription factor STAT1 (See, Gubán B. et al. 2016); increased expression of SOCS1 and SOCS3 in the psoriatic epidermis (See, Madonna et al. 2012); increases in the expression of serum vascular endothelial growth factor (VEGFA) (See, Meki A. R. et al. 2014); increased expression of transforming growth factor beta TGFB1 (See, Gambichler et al. 2013); decreased expression of the receptor for transforming growth factor beta (See, Yu H. et al. 2009) associated with increased expression of SMAD3 (See, Gambichler et al. 2013); increased expression of the monocyte chemoattractant protein-1 (See, Gillitzer R. et al. 1993); decreased expression of T-cadherin in the lesional skin of psoriasis vulgaris (Mukoyama Y. et al. 2005); increases in arachidonic acids (See, Hammarstrom S. et al. 1975); decreased levels of azelaic acid, eicosapentaenoic and docosahexaenoic acids and phosphatidyl inositol (See, Chunwei Zeng. et al. 2017); decreased levels of plasma zinc levels in patients with generalized Pustular Psoriasis (See, Dreno B. et al. 1986); significantly decreased levels of spermidine and spermine in the skin of psoriasis patients (See, Proctor M. S. et al. 1975); perturbations in Melatonin rhythms (See, Mozzanica N, et al. (1988) and many others.

Efforts for identifying molecular mechanisms and causal relationships between these imbalances indicates that most if not all imbalances are caused by inflammatory responses to dysfunctional metabolic regulation of stem cell proliferation and stem cell differentiation at the epidermis and T cells level with keratinocytes and Th-17 cells as the main culprits (See, Nestle F. O., et al. 2009 and Table 1 in this analysis).

TABLE 1
PROTEINS ASSOCIATED WITH THE PATHOLOGY OF PSORIASIS
Functional relationships between proteins can be ascertained using the String
database (Szklarczyk D, et al. The STRING database in 2017: quality-controlled
protein-protein association networks, made broadly accessible. Nucleic Acids Res.
January 2017; 45:D362-68).

| | | | | | | | |
|---|---|---|---|---|---|---|---|
| ABCA1 | | HIF1A | | | | | |
| APOA1 | CCL5 | IFIT1 | IL23 | JAK1 | NRBF2 | RBX1 | |
| ATG14 | CDH13 | IFIT2 | IL23R | JAK2 | NRF2 | SAA | TGFBR1 |
| ATG2 | CSF2 | IFIT3 | IL4 | JUN | PI3K | SMAD2 | TLR2 |
| AXIN1 | CTNNB1 | IL10 | IL5 | JUNB | PIK3C2A | SMAD3 | TNFRSF1B |
| BTRC | CUL3 | IL13 | IL7 | KEAP1 | PIK3CA | SOCS1 | TP53 |
| CCL17 | CXCL10 | IL15 | IL7R | LCAT | PIK3CG | SOCS3 | TRAF3IP2 |
| CCL18 | CXCL11 | IL17A | IMPA1 | LPA | PLA2G2A | SQSTM1 | TYK2 |
| CCL2 | CXCL9 | IL17F | IMPA2 | LPC | PLA2G2F | STAT1 | ULK1 |
| CCL22 | CXCR3 | IL17RA | IRF1 | MTMR4 | PLCE1 | STAT3 | VEGFA |
| CCL27 | CXCR4 | IL18 | IRF2 | MTOR | PPARA | STUB1 | WIPI1 |
| CCL3 | DKK1 | IL2 | IRF9 | NFE2L2 | PPARG | TFAM | ZBTB16 |
| CCL4 | GSK3B | IL21 | ISG15 | NLRP3 | PTGS2 | TFEB | RARA |

### SUMMARY

In one embodiment, a pharmaceutical composition is provided. The pharmaceutical composition includes at least one of a first group consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil; at least one of a second group consisting of phosphatidylinositol, inositol, and aspirin; and at least one of a third group consisting of 2-hydroxypropyl-β-cyclodextrin, beta-hydroxy-beta-methylbutyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus; and a pharmaceutically acceptable carrier.

In another embodiment, a method of treating a disease or the symptoms thereof in a mammal, the disease selected from the group consisting of psoriasis, arthritic psoriasis, pediatric psoriasis, plaques, acne, atopic dermatitis, Danon Disease, depressive disorders, dyspraxia, multiple sclerosis, bone loss, colitis, inflammatory bowel disease, Crohn's Disease, ulcer, rheumatoid arthritis, cardiovascular disease including atherosclerosis, cardiomyopathy, congestive heart failure or endocarditis, liver diseases including nonalcoholic fatty liver disease, nonalcoholic steatohepatitis, or primary biliary cholangitis, lung diseases including interstitial lung disease, idiopathic pulmonary fibrosis, hypersensitivity pneumonitis, sarcoidosis or asbestosis, aging associated diseases including atherosclerosis, arthritis, cataracts, osteoporosis, type 2 diabetes, hypertension, Alzheimer's disease, hearing loss, maculopathy, osteoarthritis, Parkinson's disease, periodontitis, rheumatoid arthritis or sarcopenia, post-traumatic stress disorder, autism spectrum disorders, stroke, Rett syndrome, obesity, infertility, traumatic brain injury and diseases caused by the hyperproliferation of cells including colorectal cancer, osteomyelitis, Hodgkin Disease, Bladder cancer, prostate cancer, ovarian cancer, gall bladder cancer, pancreatic cancer, esophageal cancer, liver cancer, mesothelioma or lung cancer is provided. The method includes administering to said mammal in need of such treatment an effective amount of a pharmaceutical composition of the embodiments of the present disclosure.

In another embodiment, a method of reducing pain and inflammation or the symptoms thereof in a mammal is provided. The method includes administering to said mammal in need of such treatment an effective amount of a pharmaceutical composition of the embodiments of the present disclosure.

In another embodiment, a method of reducing gene expression or systemic levels of proteins selected from the group consisting of SQSTM1, COX 2, VEGF2, IL23, II17, IL1, IL6, TNF alpha, and PON1 in a mammal is provided. The method includes administering to said mammal in need of such treatment an effective amount of a pharmaceutical composition of the embodiments of the present disclosure.

In another embodiment, a method of treating a disease or the symptoms thereof associated with increased levels of SQSTM1 or COX2 in a mammal is provided. The method includes administering to said mammal in need of such treatment an effective amount of a pharmaceutical composition of the embodiments of the present disclosure.

In another embodiment, a method of treating a disease or the symptoms thereof in a mammal, the disease selected from the group consisting of psoriasis, arthritic psoriasis, pediatric psoriasis, plaques, acne, and atopic dermatitis, the method comprising administering to said mammal in need of such treatment an effective amount of a pharmaceutical composition of the embodiments of the present disclosure.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “treating” and “effective amount”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

Inflammation is a key step in healing processes and hence treatment modalities that merely target symptoms of inflammation and not the root causes of disease have frequently been shown to impair healing. For overcoming this shortcoming, this present disclosure includes novel pharmaceutical compositions that down regulate excessive inflammation and support cellular and molecular functions involved in healing processes. These novel compositions consist of a first group of ingredients selected from the group consisting of herring roe oil, krill oil, fish oils, herring roe proteins, krill proteins, docosahexaenoic acid, docosapentaenoic acid, eicosapentaenoic acid, stearidonic acid, lithium and zinc salts of docosahexaenoic acid, docosapentaenoic acid, eicosapentaenoic acids, stearidonic acid and the various isomers of linoleic acids believed to be converted upon administration to a mammal into anti-inflammatory substances such as morensins, resolvins and defensins in combination with effective amounts of ingredients selected from a second group consisting of aspirin (believed to facilitate the conversion of the first group of ingredients into anti-inflammatory morensins, defensis and resolvins) and phosphatidyl inositol, inositol believed to support autophagy mediated reduction of pro-inflammatory signals in combination with effective amounts of a third group of ingredients believed to increase autophagy said third group including 2′5′-dideoxyadenosine, 2-hydroxypropyl-β-cyclodextrin, 6-shogaol, 6-shogaol, AB-MECA, acacetin, acetohexamide, acetyl-11-keto-boswellic acid, alexidine, all-trans-retinoic acid, ambroxol, amiodarone, AP23841, ascomycin, astaxanthin, astragalus sinicus extracts, atorvastatin, AZD08055, berberine, berberis aristata extracts, beta-hydroxy-beta-methylbutyrate, bilobalide, boswellia serrata extracts, caffeine, calpastatin, calpeptin, cannabidiol, cannabinoids, CAPE, carbamazepine, cerivistatin, cepharanthine, clonidin, curcumin, dauricine, deferolimus, diclofenac, diosgenin, doxycycline, elaidylphosphocholine, epigallocatechin gallates, esomeprazole, estradiol, everolimus, EX2044, EX3855, EX7518, fasudil, fluspirilene, fluvastatin, fulvestrant, fusaric acid, gallic acid, ganoderma lucidum extract, genkwanin, genistein, glucosamine, grayanotoxin III, GW7647, hernandezine, HU-308, hydroxytyrosol, indatralin, INK-128, isorhamnetin, isotretinoin, KU-0063794, L-690330, lanthionine ketimine, lanthionine ketimine-5-ethyl ester, lithium chloride, loperamide, lovastatin, LY303511, LY-307265, mahonia aquifolium extracts, melatonin, metformin, minoxidil, monascus purpureus, N5-methyl adenosine, NCGC758, neomycin, NF449, niclosamide, niguldipine, nimodipine, nitrendipine, oleuropein, oleuropein Aglycone, oregon grape root extracts, OSI-027, penitrem A, perhexiline, perifosine, PI 3-kinase inhibitors, pimozide, pioglitazone, plerixafor, pravastatin, pterostilbene, pyrroloquinoline quinone, quercetin, quercitrin, rapamycin, resveratrol, retinoic acid, rilmenidine, ripasudil, ritodrine, root of curcuma longa, root of polygonum cuspidatum, rosuvastatin, rosglitazone, rottlerin, SB242084, simvastatin, sirolimus, SMER-28, sodium valproate, spermidine, telmisartan, thalidezine, temsirolimus, trehalose, tretinoin, trifluoperazine, troglitazone, urolithin A, urolithin B, urolithin C, urolithin D, valproic acid salts, verapamil, vitexin, vitis vinifera extracts, WYE-125132, xestospongin B, XL765, zinc acetate, zinc aspartate, zinc carbonate, zinc citrate, zinc gluconate, zinc oxide, zinc phosphate, zinc sulfate, zotarolimus and methods related thereto.

In certain embodiments, the present disclosure also relates to methods of treating or preventing diseases or disease conditions associated with systemic inflammation mediated in part by increased expressions of interleukin 23 (IL23), interleukin 17 A (IL-17A) and dysregulation of autophagy functions characterized by increased levels of autophagy associated protein SQSTM1 (also known as P62) and dysregulation of stem cell homeostasis wherein said diseases or disease conditions are selected from the group consisting of autism spectrum disorders, skin disorders, nephritis, acne, aging associated diseases (e.g., atherosclerosis, arthritis, cataracts, osteoporosis, type 2 diabetes, hypertension, Alzheimer's disease, hearing loss, maculopathy, osteoarthritis, Parkinson's disease, periodontitis, rheumatoid arthritis, and sarcopenia), Alzheimer' s disease, atopic dermatitis, Addison disease, Basedow disease, bone loss, cardiovascular disease (e.g., atherosclerosis, cardiomyopathy, congestive heart failure, and endocarditis), colitis, Behcet's disease, Crohn's Disease, Danon Disease, depressive disorders, diabetes, dyspraxia, infertility, inflammatory bowel disease, liver diseases (e.g., nonalcoholic fatty liver disease, nonalcoholic steatohepatitis, and primary biliary cholangitis), lung diseases (e.g., interstitial lung disease, idiopathic pulmonary fibrosis, hypersensitivity pneumonitis, sarcoidosis, and asbestosis), multiple sclerosis, nephrosis, obesity, Parkinson's disease, pernicious anemia, post-traumatic stress disorder, psoriasis, renal disease, Rett syndrome, rheumatoid arthritis, stroke, systemic lupus erythematosus, traumatic brain injury, ulcer, ulcerative colitis, Wegener's granulomatosis and various diseases caused by the hyperproliferation of cells (e.g., colorectal cancer, osteomyelitis, Hodgkin Disease, Bladder cancer, prostate cancer, ovarian cancer, gall bladder cancer, pancreatic cancer, esophageal cancer, liver cancer, mesothelioma and lung cancer) cells by administering effective amounts of the novel compositions disclosed herein to a subject in need thereof. Preferred aspects of the present disclosure include combinations and pharmaceutical compositions as well as methods incorporation the embodiments of the present disclosure for the treatment of psoriasis, psoriatic associated conditions and amelioration of psoriasis symptoms.

In certain embodiments, it is contemplated that compositions disclosed herein contain ingredients frequently also found in derivative form in herbal extracts and prodrugs

Stem cell differentiation and stem cell proliferation (collectively called stem cell homeostasis), are processes involved in the renewal of tissues, skin, aging, metabolic disease, wound healing, repair of injuries to the peripheral and central nervous systems and processes associated with neurogenerative diseases and depression. It is also generally accepted that the differentiation of stem cells to a specific lineage is precipitated by a metabolic switch resulting in a shift from glycolysis to oxidative phosphorylation for generating energy (See, Mandal et al. 2011; Shyh-Changet al. 2013; Takubo et al. 2013). For example, mitochondrial functions become activated during stem cell differentiation (See, St John et al. 2005; Cho et al. 2006; Facucho-Oliveira et al. 2007; Chung et al. 2010; Prigione et al. 2010; Yanes et al. 2010; Norddahl et al. 2011). Thus, in case of neuron renewal (neurogenesis), neuronal stem cells before differentiation prefer glycolytic metabolism for energy production while postmitotic and differentiated neurons rely primarily on oxidative phosphorylation (See, Llorens-Bobadilla et al. 2015; Shin et al. 2015; Agostini et al. 2016; Khacho et al. 2016; Beckervordersandforth et al. 2017b). Likewise, CD4+ T cells, which are a proinflammatory cell subset, preferentially produce ATP through glycolysis, whereas cells with an anti-inflammatory lineage, such as memory and regulatory T cells, favor mitochondrial ATP generation (See, Olenchock B. A. 2017). The metabolic switch separating proliferating and differentiating stem cell populations requires the coordinated regulation of various mitochondrial functions (See, Homem et al. 2014; Stollet al. 2015). For example, increases in mitochondrial respiratory capacity and mitochondrial mass observed during stem cell differentiation are associated with increases in mitochondrial biogenesis and activation of mitochondrial metabolism (See, Wang et al. 2010, 2011, Piccoliet al. 2005; Chen et al. 2008; Zhang et al. 2011). Since increases in mitochondrial biogenesis are known to increase levels of reactive oxygen species (ROS) which are known to cause damage to mitochondrial DNA, sustaining this function requires additional mechanisms for protecting differentiating stem cells from damage inflicted by reactive oxygen species by repairing damage to mitochondrial DNA and replacing damaged mitochondria (See, Wang et al. 2010, 2011). When repair and removal of damaged mitochondria and cellular component through processes termed mitophagy and autophagy (See, Nature Editors 2018) become impaired, cellular senescence programs become activated. Thus, it is well known that activation of cellular senescence is associated with development of aging associated disease (See, Wang et al. 2010). Accordingly, pharmaceutical composition capable of restoring, inducing or activating autophagy functions are anticipated to have utility for treatment of aging associated disease.

In skin, differentiating keratinocytes which are the predominant cell type in the epidermis, undergo a selective form of nucleophagy (See, Han P. et al. 2016) and failure in the initiation of nucleophagy is associated with parakeratosis (See, Olufolake A. et al. 2016) which is usually seen in the plaques of psoriasis and in dandruff.

Cellular processes modulating autophagy and reactive oxygen species (ROS) generation (See, Komatsu M. et al. 2010; Li. L. et al. 2015; Reuter S. et al. 2010) are cross regulated by interacting protein networks. While, autophagy was considered for a long time to be a non-selective pathway for degradation (See, Rinaldo Florencio-Silva et al. 2017), this process can be selectively targeted for specific organelle degradation by regulating levels and activities of p62 protein also termed SQSTM1. Thus, p62 binds ubiquitinated proteins and transports them to the interior of autophagosomes where it is ultimately degraded. Balancing synthesis and degradation of p62 protein has important regulatory functions. Observing an increased expression of P62/SQSTM1 in psoriasis indicates that psoriasis is associated with a dysfunction of the autophagic degradation process. For identifying mechanism involved in the dysregulation of autophagy in psoriasis, we used a new technology termed information flow analysis (See, Fliri A. et al. 2018) for identifying key segments of the protein network involved in this dysregulation.

Towards this end an analysis of protein interaction networks regulating cellular processes associated with the pathogenesis of psoriasis, identified molecular processes regulating the production of mediators of inflammatory signaling such as for example, the nuclear factor kappa-light-chain-enhancer of activated B cells (NFKB), the transcription factor STAT1, hypoxia induced factor alpha and, among many others, transcription factors NRF2 and NRBF2 and the enzyme COX2 known to be differentially expressed and/or regulated in psoriasis. This analysis further identified that the autophagy adaptor protein SQSTM1 (also termed p62), which is overexpressed in psoriatic skin, not only regulates the generation of inflammatory cytokines in primary human keratinocytes (See, Hye-Mi Lee. et al. 2010), but also, the activity of NFK-b and NRF2 which modulate the expression of the arachidonic acid (AA) metabolizing enzyme COX2 (See, Bakry O A et al. 2015) and the expression of the vascular endothelial growth factor VEGFA. Both of these proteins are overexpressed in psoriasis (See, Kweider N et al. 2011). The increased expression of VEGF in psoriatic skin causes an increased expression of monocyte chemoattractant protein-1 CCL2 (See, Marumo T. et al. 1999) which, in turn, precipitates an infiltration of innate immune cells into affected skin and causes the excessive angiogenesis and vasodilation observed in psoriatic tissue (See, Matkar, P. N et al. 2017). The relevance of these processes in the pathogenies of psoriasis is highlighted by the observation that expression levels of NFKB and COX2 correlate with psoriasis severity scores (See, Bakry O A et al. 2015). The overexpression of COX2 (See, Bakry O. A. et al. 2015) in combination with the observed increases in Arachidonic acid levels in psoriasis (See, Hammarstrom S. et al. 1975) leads to the excessive production of proinflammatory eicosanoids aggravating skin inflammation. Moreover, COX2 also mediates the conversion of the omega 3 fatty acids, eicosapentaenoic acid (EPA) and docosahexaenoic acid (DHA) into anti-inflammatory mediators known as resolvins, marensisns and protectins and increases the expression of the autophagy inhibitor BCL2 (See, Xin-Hua Liu et al. 1998; Zhou L. et al. 2015). Thus, this dual role marks COX2 as one of the key target enzymes for psoriasis disease intervention (See, Corinne Joffre 2014). Thus, pharmacological compositions substances capable of modulating the balance between proinflammatory and anti-inflammatory signal generation are not only useful for treatment of psoriasis but also for treatment of other inflammatory diseases associated with a dysregulation of the production of pro and anti-inflammatory signal mediators. Indicating that the balance between pro and anti-inflammatory signal plays a key role in generating the pathology of psoriasis is the observation that levels of the proinflammatory arachidonic acid are increased and levels of the anti-inflammatory fatty acids EPA and DHA are decreased in psoriasis (See, Chunwei Zeng. et al. 2017). This shift in fatty acid ratios tips the balance towards COX2 mediated arachidonic acid metabolism generating proinflammatory eicosanoids and decreases the production of anti-inflammatory resolvins, morensisn protectins derived from the metabolism of EPA and DHA by COX2. Moreover, the observed increased expression of COX2 in psoriasis is expected decreases Beclin 1-dependent autophagic cell death which explains, in part, the observed apoptosis resistance of psoriasis associated keratincocytes (See, Xin-Hua Liu et al. 1998). Thus, pharmaceutical composition that increase levels of EPA and DHA in psoriasis such as for example herring roe oil and krill oil in combination with substances that lower COX expression levels such as for example stearidonic acid (See, Horia E. et al. 2005) or substances that redirect COX 2 activity towards EPA and DHA metabolism such as for example aspirin (See, Serhan C.N. et al. 2002) will not only be useful for the treatment of psoriasis but also for treatment of many other diseases with inflammatory components such as for example, depressive disorders, multiple sclerosis, bone loss, colitis, inflammatory bowel disease, Crohn's Disease, ulcer, rheumatoid arthritis, cardiovascular disease (e.g., atherosclerosis, cardiomyopathy, congestive heart failure, endocarditis), renal disease, liver diseases (e.g., nonalcoholic fatty liver disease, nonalcoholic steatohepatitis, and primary biliary cholangitis), lung diseases (e.g., interstitial lung disease, idiopathic pulmonary fibrosis, hypersensitivity pneumonitis, sarcoidosis, and asbestosis), aging associated diseases (e.g., atherosclerosis, arthritis, cataracts, osteoporosis, type 2 diabetes, hypertension, Alzheimer's disease, hearing loss, maculopathy, osteoarthritis, Parkinson's disease, periodontitis, rheumatoid arthritis, and sarcopenia), post-traumatic stress disorder, autism spectrum disorders, stroke, Rett syndrome, Parkinson's disease, Alzheimer's disease, diabetes, obesity, infertility, traumatic brain injury, skin disorders, atopic dermatitis, acne and various cancers.

Focusing on molecular mechanism relevant to skin health is a process termed nucleophagy which is a specialized from of autophagy that extracts nuclei form mitochondria of terminal differentiated keratinocytes. This process is constitutively active in the epidermal granular layer of the skin and is required for normal keratinocyte homeostasis. Recent findings by Olufolake (See, Olufolake A. et al. 2016) indicate that impairment of this process leads to an accumulation of P62 in psoriatic tissue and that P62 accumulation may play a key role in the pathogenesis of psoriasis.

Thus, in keratinocytes levels of SQSTM1 are regulated, in part, by interactions of acute-phase serum amyloid protein A (SAA1) and the cytokine IL17 with the Toll-like receptor 2 (TLR2) which is known to plays a key role in the regulation of innate immunity and autophagy (See, Chang C.P.et al. 2013). Ascribing insignificance to this regulation in psoriatic skin, levels of SQSTM1, SAA1, IL17 and TLR2 are increased in psoriasis (See, Yu N et al. 2015; Begon, E. et al. 2007; Jin-Man Kim et al. 2013) and are associated with a dysregulation of autophagy (See, Hye-Mi Lee. et al. 2010). Regulation of autophagy in psoriasis involves in part the dynamic phosphorylation and dephosphorylation of Glycogen synthetase beta 3 (GSKB3) (See, Zhang et al. 2015). This dynamic GSKB3 regulation is co-regulated by modulating the levels of reactive oxygen species (ROS) which are modulated by actions of inflammatory signals and ligands. Many of these regulatory signals are altered in psoriasis. For example, spermidine, a polyamine compound found with various metabolic functions is known to activates autophagy (See, Morselli, E. et al. 2011) and its levels are decreased in psoriasis (See, Proctor, M. S. et al. 1975). In contrast. the pro-inflammatory cytokine IL17, known to inhibit autophagy by triggering trigger the release of reactive oxygen species (ROS) is upregulated in psoriasis (See, Liu, H. et al. (2013). Moreover, perturbations of HDL cholesterol levels and lipid compositions known to impair autophagy are also observed in psoriasis (See, Varshney P et al. 2018, Haruna, Kunitaka. et al. 2008). Thus, the combination of these events leads to the impairment of autophagy mediated SQSTM1 degradation, which is manifested by elevated levels of SQSTM1 in psoriasis (See, Jin-Man Kim et al. 2010). Supporting the premise that autophagy dysfunction mediated elevation of SQSTM1 levels is a key step in the pathogenesis of psoriasis is the observation that the silencing of SQSTM1 expression through RNA interference decreases inflammatory cytokine production and the hyper proliferation of keratinocytes (See, Hye-Mi Lee. et al. 2010; Olufolake Akinduro et al. 2016).

One of the prominent proteins regulating autophagy is MTOR which modulates the activity of Nuclear receptor-binding factor 2 (NBRF2) which, in turn, regulates the activity of PIK3 phosphatidylinositol kinases. For example, the dephosphorylation of NRBF2 promotes Ptdlns3K lipid kinase activity and autophagy flux, whereas NRBF2 phosphorylation blocks these processes.

Yet another mechanism for finetuning autophagy in psoriasis involves the regulation of autophagosome formation by controlling levels of the autophagy associated ATG14L protein. This flavor of autophagy regulation is affected by interactions of ligands with G Protein Coupled Receptors via Ptdlns(4,5)P2 mediated signaling pathways (See, Tan X. et al. 2016). For example, the activation or inactivation of GPC receptors involved in this mode of autophagy regulation increase or decrease levels of the Zinc transcription factor ZBTB16, which, in turn, control the degradation of Atg14L and by this mean regulate autophagy. This autophagy control mechanism is further finetuned through the phosphatidylinositol 3-kinase (PIK3) mediated phosphorylation of Glycogen synthetase beta 3 (GSKB3). Thus, the inhibition of phosphatidylinositol 3-kinase causes the activation of GSKB3, which in turn, decreases levels of the Zinc transcription factor ZBTB16 and, by increasing levels of ATG14L, increases autophagic flux. Thus, the observed overexpression of phosphatidylinositol 3-kinase in psoriasis (See, Zhang X et al. 1999) is expected to inhibit autophagy flux. Again, this observation directly implicates the regulation of autophagy in the pathogenesis of psoriasis. Accordingly, inhibitors of phosphatidylinositol 3-kinase (See, Ito K. et al. (2007) such as for example, quercetin, are expected to counteract effects of phosphatidylinositol 3-kinase overexpression in psoriasis and, by activating autophagy, are useful additions to pharmaceutical compositions for treatment of psoriasis.

An atypical mechanism of autophagy regulation is the sequestration of SQSTM1/p62 involving the inhibition of cyclic AMP phosphodiesterase-4A4 (See, Christian F. et al. 2010). P62 sequestration is anticipated to reduce interaction of p62 with KEp1 and hence reduce proinflammatory signal generation. Thus, this mechanism could contribute to anti-inflammatory properties and therapeutic utility of PDE4 inhibitors in psoriasis (See, Moustafa F. et al. 2014).

HDL particles have complex structures and multiple regulatory functions (See, Rosenson R S. et al. 2016). In that, ApoL1 and ApoL6 are crucial regulators of cellular homeostasis and ApoL1, when overexpressed intracellularly, induces autophagy in all cell types (See, Hu C. A. A. et al. (2012). Noteworthy, the observed decreased levels of AP L1 in psoriasis (See, Seishima M. et al. 1994) are anticipated to negatively affect autophagy. The significance of this contribution to the pathogeneis of psoriasis can be gleaned from the observation that anti-psoriatic therapy significantly improves serum lecithin-cholesterol acyltransferase activity and autophagy (See, Holzer M. et al. 2013; Pietrzak A. et al. 2010; Varshney P et al. 2018). These observations indicate that substances affecting functions of HDL particles in combination with substances affecting autophagy activity are useful for treatment of psoriasis.

Autophagy processes are also co-regulated by the modulation of phospholipase activities via HDL (See, Bae, E. J.et al. 2014); Zhu S. et al. 2016). For example, PLA2G2F, which is a functionally orphan-secreted phospholipase A2, is expressed in the suprabasal epidermis, and regulates skin homeostasis. PLA2G2F preferentially hydrolyzes ethanolamine plasmalogen-bearing docosahexaenoic acid secreted from keratinocytes and to give rise to protectin D1 and 9S-hydroxyoctadecadienoic acid. Deletion of PLA2G2F protects mice from developing psoriasis, contact dermatitis, and skin cancer. Primary keratinocytes of PLAG2F minus mice show defective differentiation and activation (See, Yamamoto Kei et al. 2015). Addition of ethanolamine lysoplasmalogen which derived from the PLA2G2F activity abolishes effects of PLAG2F deletion both in vitro and in vivo. Conversely, the overexpression of PLA2G2F lead to development of psoriasis-like epidermal hyperplasia. The expression of PLA2G2F in keratinocytes can be induced by calcium or IL-22. Accordingly, pharmaceutical compositions capable of down regulating phospholipase activity such as PLA2G2F are useful for treatment of psoriasis and inflammatory disease or conditions associated with overexpressed or overactive PLA2G2F.

A MTOR and glycogen synthase kinase 3 β independent mechanism of autophagy regulation relevant to psoriasis is the modulation of mono phosphatase activities (IMP) which regulate the conversion of phosphatidylinositol into monophosphate derivatives and lead to a depletion of free inositol and phosphatidylinositol levels. (See, Sovan S. et al. 2005). Thus, Lithium, which is a known inducer of psoriasis inhibits inositol monophosphatases, and this inhibition causes a depletion of free inositol and myo-inositol-1,4,5-triphosphate (IP3) levels. This lithium effect can be counteracted by pharmacologic treatments that increased IP3 levels (See, Sovan S. et al. 2005; Rosivatz E. 2011). Thus, clinical studies using up to 6 grams of inositol daily have been shown to reduce symptoms of lithium induced psoriasis (See, Allan S. J. et al. 2004). The role of inositol and phosphatidylinositol levels in regulating inflammation can be inferred from observations indicting that phosphatidyl inositol (See, Van Dieren et al. 2011) and myoinositol (See, Rysz J. et al. 2006) have direct anti-inflammatory activity. Thus, the identification of phosphatidylinositol and inositol levels, as regulators of autophagy (See, Sarkar S. et al. 2006) explains the discrepancy of why lithium, which is an autophagy activator also induces psoriasis. Thus, by impairing monophosphatases Lithium lowers levels of phosphatidyl inositol and inositol levels which are already lower in psoriasis patients when compared to controls and, in doing so impairs autophagosome formation (See, Chunwei Zeng. et al. 2017; Axe E. L. et al. 2008). Accordingly, Pharmacological means that restore this regulatory function by adding phosphatidylinositol or inositol to pharmaceutical compositions are useful for treatment of psoriasis. Supporting this function is the observation that diets with phosphatidylinositol supplementation significantly decrease proinflammatory cytokine serum levels (See, Inafuku M. et al. 2013).

Noteworthy, dysregulation of autophagy is also observed in psoriasis associated comorbidities such as depression, bone resorption and Gaucher disease. For example, defect control of p62 levels caused by autophagy dysregulation play a role in genesis of depression (See, Toshifumi T. et al. (2018); bone resorption (See, Daroszewska A. et al. 2011) and Gaucher disease, which, like psoriasis, is characterized by markedly low HDL cholesterol levels (See, Stein, P.et al. 2011) and elevated levels of the autophagic adaptor p62 triggering increased inflammasome activation (See, Aflaki Elma et al. 2016).

The relationships between ROS levels and development of psoriasis, in turn, has been examined in detail in mice models of this disease (See, Khmaladze et al. 2015; Khmaladze et al. 2014; Guerard S et al. 2016). Thus, these model studies found that mice with deficient capacity for generating reactive oxygen species suffer exacerbation of disease symptoms while the restoration of ROS production ameliorates disease See, Khmaladze I et al. 2014; Guerard Set al. 2016).

In addition to discovering that ROS-levels affect severity of psoriasis, these studies also found that interleukin-17 levels which are increased in psoriasis (See, Soderstrom C, et al.2017), play a key role in disease development and that the neutralization of IL-17A completely blocked psoriasis pathogenesis (See, Khmaladze I et al. 2014). Subsequent clinical trials with IL 17 antibodies confirmed this cause effect relationship by achieving nearly complete remission of the disease.

IL-17, promotes the release of reactive oxygen species in cells expressing the IL17 receptor (IL17R) such as for example keratinocytes and Th17 cells. Likewise, acute-phase protein serum amyloid A (SAA) which is overexpressed in Psoriasis has been reported to be an inducer of ROS, to trigger ROS dependent expression of the inflammasome regulator NLRP3, the expression of the proinflammatory cytokine IL1-beta in psoriatic keratinocytes (See, Martinon F. 2010) and, by increasing the secretion of IL23 in T cells, also increase levels of IL17 (See, He R. et al. 2006; Papp K A. et al. 2017).

SAA mediated activation of proinflammatory signals is counteracted by High-Density Lipoprotein (HDL) levels which upon binding to SAA reduces the secretion of proinflammatory cytokines (See, Zhu S et al. 2016). Since functional HDL levels are reduced in psoriasis and levels of Omega 3 fatty acids such as Eicosapentaenoic acid and Docosahexaenoic acid which are known to repair dysfunctional HDL (See, Davidson M H. 2013) are also reduced in psoriasis, the increased levels of SAA in psoriasis remain unopposed. The resulting escalation of IL17 mediated inflammatory responses and reactive oxygen production, in turn, cause the inhibition of glycogen synthase kinase 3 (GSK3B) which is a key regulator of mitochondrial energy production (See, Plyte et al. 1992) and the Wnt-pathway which is involved in stem cell homeostasis (See, Gudjonsson, Johann E. et al. 2010). Thus, besides its proinflammatory role among the key effects induced by IL17 is the inhibition of the activity of glycogen synthase kinase 3 (GSK3B) which in its active form down regulates of the degradation of p-catenin (CTNNB1). Therefore, increased IL 17 levels lead to the accumulation of Catechin in the cytosol which, upon translocation to the nucleus, increases the expression of a wide range of target genes including the Wnt4 protein which is involved in mesenchymal-to-epithelial transition (See, Kispert et al. 1998, Sutherland, C.et al. 1993) and neurodegeneration (See, Cerpa W. et al. 2009).

Thus, compositions including substances capable of reducing IL17 expression and Wnt signaling such as for example, Melatonin and Aspirin in combination with substances capable of down regulating SQSTM1 levels and restoring COX 2 mediated imbalances between pro and anti-inflammatory signal mediators such as EPA and DHA will be useful for treatment of psoriasis and neurodegenerative disease.

In addition, the inhibition of glycogen synthase kinase 3 (GSK3B) activity within the AMPK complex (functioning as an energy sensor) also increases the expression of peroxisome proliferator-activated receptors PPARA, PPARG and PPARD/B and precipitates the activation of mitochondrial biogenesis. (See, Theeuwes. et al. 2018). Thus, the activation of the PPARA subtype reduces triglycerides and regulates energy homeostasis, activation of the PPARG subtype causes insulin sensitization and enhances glucose metabolism, while the activation of the PPAR delta subtype enhances fatty acid metabolism (See, Pitchai Balakumara et al. 2007).

Activation of mitochondrial biogenesis, in turn, leads to further increases in ROS levels which is consistent with the observation that keratinocytes have increased mitochondrial respiration coupled to ATP synthesis. Since elevation of ROS levels exposes mitochondria to damage, ROS levels are generally adjusted through the modulation of phosphatidylinositol 3-kinase (PIK3CA) activities. For example, inhibition of PIK3CA dampens the phosphorylation of glycogen synthase kinase 3, decreases ROS levels and reverses the induction of mitochondrial biogenesis (See, Bai, Fuhai. et al. 2017) while activation does the opposite. Since IL10 is known to activate phosphatidylinositol 3-kinase (PIK3CA), the observed down regulation of IL-10 levels in psoriasis would decrease the effectiveness of this ROS level reducing mechanism and support the high respiratory activity in keratinocytes of psoriasis patients (See, Keith W. et al. 2001; MacGarvey. et al. 2012). Supporting the contribution of IL10 reduction to the pathogenesis of psoriasis is the observation that a specific subset of IL-10-producing regulatory B cells so-called Bregs, which inhibit differentiation of Th1 and Th17 cells are significant decreased in psoriasis (See, Athanasios Mavropoulos et al. 2017). Contribution this ROS regulating mechanism to pathogenies also explains the role of elevated levels of the branched chain amino acid leucin in psoriasis (Edwards et al. 2015; Yan, J. et al. 2017) which is known to activate phosphatidylinositol 3-kinase (PIK3CA) (See, Peyrollier K et al. 2000; Sun et al. 2009) and enhances respiratory activity. Likewise, the observed increased expression of the suppressors of cytokine signaling SOCS1 and SOCS3 in psoriatic keratinocytes sustains the activation of the PIK3CA/AKT pathway and suppresses keratinocyte apoptosis (See, Madonna et al. 2012). Thus, inhibition or down regulation of phosphatidylinositol 3-kinas with pharmacological means is anticipated to be useful for treatment of psoriasis.

Lastly direct evidence of a dysregulation of mitochondrial Wnt pathway signaling in pathogenesis of psoriasis is provided by Lithium, which is an inhibitor of glycogen synthase kinase 3 (See, Hampton P. J. et al. 2012) and one of the most common causes of drug-induced psoriasis. Thus, lithium induces the proliferation of keratinocyte by activating Wnt signaling pathway (See, Hedgepeth et al. 1997), increasing the release and expression of TNF (See, Giambelluca, Miriam S. et al. 2014) and inhibiting monophosphatase mediated regulation of autophagy activity by decreasing phosphatidylinositol pools. Supporting keratinocyte proliferation at increased ROS levels, in case of mitochondrial dysfunction, damaged mitochondria release the phosphatase family member 5 (PGAM5) into the cytosol, which upon release dephosphorylates AXIN1-bound p-catenin (CTNNB1), activates Wnt/p-catenin signaling and triggers replacement of dysfunctional mitochondria. This autophagy independent replacement mechanism of defunct mitochondria is supported by the overexpression of the transcription factor Stat3 in psoriasis (See, Andres R.M. et al. 2013) which, has been shown to rescue impaired mitochondrial function (See, Wegrzyn et al. 2009).

The role of TGFβ/Smads signaling in the pathogenesis of psoriasis is supported by the psoriasis treatment using UVB which leads to the induction of the antagonist Smad 7 (See, Quan et al. 2002) and explains the antiproliferative effects of 1a, 25-dihydroxyvitamin D3 in model systems which seem in part to be mediated by TGFp and Smad signaling (See, Yanagisawa et al. 1999); however, since Vitamin D3 is known to upregulate IL17 production (See, Reda, R. et al. (2015) it is conceivable that Vitamin D3 could diminish the efficacy of anti-inflammatory psoriasis compositions.

Thus, this system wide cause-effect analysis indicates that psoriasis is caused in part by perturbations in lipid homeostasis leading to lower HDL levels, decreases in LDL size (See, Nehal N.et al. 2012) increases in Lipoprotein A (LPA) levels (See, Latha, K. P.2009) and decreases in apo A-I levels (See, Seishima, M. et al. 1994) which precipitate either directly or indirectly impairment of autophagy characterized by increased autophagosome formation, increased autophagy flux, and increases in P62 levels. Increases of P62 levels as observed in psoriatic keratinocytes, leads to increasing NRF2 mediated transcription and expression of proinflammatory signals including COX2. Increased levels of acute-phase protein serum amyloid A (SAA) in psoriasis coupled with (See, Yu N et al. 2015) the observed upregulation of the SAA target receptor TLR2 in psoriasis (See, Yu N et al. 2015) which may be exacerbated by shifts in microbiome communities (See, Alekseyenko, A. V. et al. 2013). These factors either alone or in combination stimulate the production of inflammatory factors such as IL 23, IL17, NRF2, STAT3 (See, Andres R. M. et al. 2013, O'Reilly S. et al. 2014) in skin. Thus, the observed lower HDL levels in psoriasis patients augment this pro-inflammatory signaling cascade by decreasing the down regulation of SAA mediated inflammatory processes by diminishing the binding of SAA to HDL. Moreover, the observed decreased levels of eicosapentaenoic acid and docosahexaenoic in psoriasis and increased levels of arachidonic acid (See, Chunwei Zeng. et al. 2017) further diminish the impact of this anti-inflammatory process by (1) decreasing the ability of eicosapentaenoic acid (EPA) and docosahexaenoic acid (DHA) to restore functional competent serum lipids particle sizes (See, a Mori, Trevor A. et al. 2000; Chunwei Zeng. et al. 2017); (2) decreasing the production of COX2 mediated anti-inflammatory resolvins, protectins and marensins derived from DHA and EPA (See, Schmid M. 2015) and (3) increasing the production of proinflammatory eicosanoids derived from arachidonic acid inducing pain, fever and vasodilatation. Moreover, the flood of cytokines produced by this proinflammatory response cascade exacerbates disease by stimulating the release and the production of reactive oxygen species which, in turn, by causing the inhibition of glycogen synthase kinase-3β (GSK-3β) and the activation of the canonical WNT pathway, stimulate the hyperproliferation of apoptosis resistant keratinocytes. Noteworthy, processes involved in the pathogenesis of psoriasis play a role in many diseases and conditions with inflammatory components such as diabetes, neuro degenerative disease, cancer and aging (See, Valee A. et al. 2018; Pareek T. K. et al. 2011) and diseases mentioned in references cited in in this background information.

Aspects of the present disclosure include novel combinations, pharmaceutical compositions and methods of treatment making use thereof for ameliorating symptoms of inflammatory disease including psoriasis consisting of omega 3 fatty acids and their derivatives in combination with substance capable of facilitating autophagy activation and reducing generation of proinflammatory signals. These are novel treatment combinations.

Concerning pharmacological compositions comprising of eicosapentaenoic acid and docosahexaenoic acid for treatment of psoriasis, the Psoriasis Area and Severity Index is correlated with circulating levels of these fatty acids and that levels of eicosapentaenoic and docosahexaenoic acids are decreased in patients with psoriasis while mono unsaturated fatty acids and arachidonic acid are increased. Moreover polyunsaturated phospholipids and omega 3 fatty acids isolated for example from Herring roe and Krill oil can be capable of affecting the regulation of stem cell proliferation and differentiation. These beneficial effects may be attributed in part to the ability of these omega 3 fatty acids to activate peroxisome proliferator-activated receptors alpha and gamma and the process termed mitochondrial biogenesis. Moreover, omega 3 fatty acids can have potent anti-inflammatory activities. Specifically, eicosapentaenoic acid (EPA) containing supplementation may improve treatment outcomes in patients with rheumatoid arthritis treated with anti-TNF modalities (See, Jeffery, 2017). EPA has also been shown to increase in the serum levels and activity of Paraoxonase 1 (See, Golzari M. H.2017) which is an antioxidant enzyme that attenuates the production of the pro-inflammatory monocyte chemoattractant protein-1 (MCP-1) (See, Marsillach, J.et al. 2009) and which is decreased in psoriasis (See, Ferretti G. et al. 2012). Since Paraoxonase 1 may play a role in regulating inflammatory responses involved in cardiovascular diseases and diabetes, pharmacological compositions described in the present disclosure and containing EPA may not only have utility for treatment of psoriasis but also for diabetes, liver diseases, arthritis and cardiovascular disease including atherosclerosis.

Moreover, eicosapentaenoic acid in combination with inositol has shown promise for reducing symptoms of depression and bipolar disorders (See, Wozniak J. et al. (2015).

In addition, studies with dodecahexaenoic acid in experimental models of autoimmune encephalomyelitis, found that dodecahexaenoic acid may reduce the number of IFNγ- and IL-17-producing CD4+ T cells (See, Kong et. Al. 2011) and expression levels of proinflammatory cytokines IL-6 and IL-23. In addition, Docosahexaenoic acid may decrease the expression levels of 1L17 and protects against inflammation by modulating IL-17/IL-23 signaling (See, Qin 2014). Moreover, these studies also found that omega 3 poly unsaturated fatty acids reduce clinical arthritis scores, ankle thickness, inflammatory cell infiltration and bone destruction that may indicate that pharmaceutical compositions containing omega 3 poly unsaturated fatty acids have utility for treatment arthritis, osteoarthritis and osteoperosis.

Studies elaborating molecular mechanism of actions of these effects found that EPA may inhibit the production of proinflammatory eicosanoids from arachidonic acid by inhibiting the activity of delta 5-desaturase, involved in the conversion of dihomo γ-linolenic acid into arachidonic acid and to inhibit the activity of phospholipase A2 (PLA2) which is involved in the release of Arachidonic acid from cell membranes. Eicosapentaenoic acid (EPA) may reduce both the proportion of AA and the production of proinflammatory eicosanoids derived from AA. Likewise, the docosanoids, resolvins and neuroprotectins (also termed Rvs and NPD1), derived from metabolism of EPA and DHA involving COX 2 may possess potent anti-inflammatory properties at physiologic dose ranges (picomolar-nanomolar) (See, Shogo T. et al. 2014). Moreover, docosahexaenoic acid and aspirin may exhibit synergistic neuroprotective effects (See, Fu Y. et al. 2017) and these natural anti-inflammatory substances may stimulate the clearance of apoptotic cells and inflammatory debris generated by macrophages, inhibit the expression of the proinflammatory cytokines, and block the infiltration of neutrophils in affected tissues. Moreover, EPA+DHA supplementation may reduce platelet aggregation, improve arterial endothelial function, and lower triglycerides and blood pressure (See, Kris-Etherton P. m. et al. 2002) and in combination with low dose aspirin significantly may improve platelet functions (See, Block, Robert C. et al. 2012). Mediating these beneficial may effect are cell-surface G-protein coupled receptors such as for example, GPR32 and ALX/FPRL2 for RvD1 and chemoattractant receptor 23 for resolvin E1. neuroprotectin D1 in turn, may exhibit also potent anti-inflammatory activity, inhibits leukocyte infiltration and the production of IL-1 by stimulated glial cells in the brain that may indicate that these substances have utility in treatment of diseases associated with neuroinflammation. Thus, neuroprotectin D1 may sustain neuronal function and protects synapses and circuits in the brain. It may also protect photoreceptors from death by promoting the expression of antiapoptotic proteins, inhibits choroidal neovascularization and induces regeneration of corneal nerves. In addition, hempseed oil containing high levels of alpha linoleic acid has been shown to improve symptoms of atopic dermatitis (See, Callaway J. et al. (2005).

Concerning additional substances, McMillan et al. referred in 1983, to psoriatic patients with more extensive skin involvement have lower levels of zinc than patients with minimal involvement. Thus, zinc may have potent anti-inflammatory activity and reduces the production of IL-17 in a dose-related fashion (See, Toghianifar, Nafiseh et al. 2015; Randa Reda et al. 2015) and hence reduced levels of zinc in psoriasis may diminish this anti-inflammatory effect. Thus, pharmaceutical compositions, containing safe doses of a bioavailable zinc salt, by remedying this zinc deficiency, may be useful for treatment of psoriasis.

Likewise, levels of phosphatidylinositol, which is one of the lipid components constituting herring roe and krill oil may be decreased in psoriasis patients. This phospholipid may be capable of repressing IL-17 and interferon gamma production without interfering in functions of regulatory T-cells (See, van Dieren 2011; Wu et al. 2016). Moreover, phosphatidylinositol which is synthesized in the endoplasmic reticulum (ER) is not only a key component for signal transduction and autophagosome formation (Balla et al. 2009; Kim et al. 2011), but also for anchoring T-cadherin, which is a regulator of keratinocyte proliferation, to the cell membrane via a glycosyl-phosphatidylinositol anchor. Dysregulation of these functions in psoriasis are indicated by the accumulation of autophagy associated P62 and decreased expression levels of T-cadherin and phosphatidylinositol in the skin of patients with psoriasis (See, Mukoyama y et al. 2005; Chunwei Zeng et al. 2017). Thus, pharmaceutical compositions that would remedy the phosphatidyl inositol deficit will be useful for treatment of psoriasis.

Several molecules inducing or promoting autophagy are available. By “induce”, or “promote” or “activate” autophagy is intended to refer to the occurrence of autophagy in a cell population increased by at least 10% when compared to the autophagy measured in absence of the molecule in the same conditions. The method suitable for measuring autophagy is detailed below and in the example section. In a preferred aspect, the capacity to induce or promote autophagy is determined by the method disclosed in Orvedahl et al. (2011, Nature, 480, 113-117, the disclosure of which is hereby incorporated herein by reference in its entirety).

Dalli, J. et al. (2013) may refer to the metabolite resolvin D3 produced by the action of COX2 enzymes on docosahexaenoic acid in presence of aspirin (See, Serhan C. N. et al. 2002) can potently regulate neutrophils and may reduce murine peritonitis and dermal inflammation; however, Dali et al. does not disclose the enhanced utility for treatment of psoriasis resulting from the addition of inositol, phosphatidylinositol, aspirin and autophagy activators to a pharmaceutical composition for treatment of psoriasis and diseases associated with aging and inflammatory conditions.

U.S. Pat. No. 6,403,656 may refer to the use of ppar-gamma activators for treatment of psoriasis, eczema, lichen planus, skin lesions associated with lupus, dermatitides, seborrho or solar dermatitis, keratoses, senile, actinic, light-induced or follicular keratosis, common acne, keloids, nevi, warts, and ichthyoses; however, U.S. Pat. No. 6,403,656 does not disclose the use of ppar gamma activators in combination with the first and second group of ingredients of the disclosure.

JP2017197472 (A) 2017-11-02 may refer to the utility of docosahexaenoic acid and eicosapentaenoic acid as inhibitors of II17 production and the utility of composition containing these substances for treatment of psoriasis; however, JP2017197472 does not disclose the utility of a pharmaceutical composition for treatment of psoriasis resulting from the addition of inositol, phosphatidylinositol, aspirin and autophagy activators to a pharmaceutical composition for treatment of psoriasis and diseases associated with aging and inflammatory conditions.

JPH07267856 may refer to the utility of docosahexaenoic acid as immunosuppressant useful for treatment of psoriasis; however, JPH07267856 does not disclose the utility of a pharmaceutical composition resulting from the addition of inositol, phosphatidylinositol, aspirin and autophagy activators for treatment of psoriasis and diseases associated with aging and inflammatory conditions.

Undurti N D, et al. (2016) may refer to the combination of aspirin with essential fatty acids such as eicosapentaenoic acid and docosahexaenoic acid is superior to aspirin alone to prevent or ameliorate sepsis or acute respiratory distress syndrome; however, Undurti N D, et al. does not disclose the utility of a composition for treatment of psoriasis resulting from the addition of inositol, phosphatidylinositol, and autophagy activators to a pharmaceutical composition for treatment of psoriasis and diseases associated with aging and inflammatory conditions.

US2017014431 may refer to compositions comprising hydrophobic derivatives of aspirin and/or hydrophobic derivatives of sesamol in combination with eicosapentaenoic acid (EPA) and docosahexaenoic acid (DHA), for reducing chronic low-level inflammation associated with chronic conditions including obesity, metabolic syndrome, diabetes, cardiovascular disease, cancer, auto-immune disorders, ocular and neurological disorders in a mammal; however, US2017014431 does not disclose the utility of a pharmaceutical composition containing phosphatitylinsoitol or inositol in combination with an autophagy activator for treatment of psoriasis, for example the addition of sesamol, which is an inhibitor and not activator of autophagy (See, Zhigang L. et al. 2017), to compositions disclosed in US2017014431 or that such an addition would be expected to decrease beneficial effects of aspirin, eicosapentaenoic acid (EPA) and docosahexaenoic acid (DHA) combinations for treatment of psoriasis and diseases associated with inflammation (See, Yazi Li et al. 2017; Ni-Tao Cheng et al. 2017).

GB2409644 (A) -2005-07-06 may refer to a composition consisting of Eicosapentaenoic acid with substantially no docosahexaenoic acid and caffeoyl esters for treatment of psoriasis. However, while caffeoyl esters are expected to increase autophagy and to be useful for treatment of skin disorders (See, Dziato, M. et al. (2016), GB2409644 does not disclose the utility of a composition for treatment of psoriasis resulting from the addition of inositol, phosphatidylinositol, and aspirin to a pharmaceutical composition for treatment of psoriasis and diseases associated with aging and inflammatory conditions.

WO2006111633 (FR20050003827 20050418) may refer to lecithin compositions derived from fish oil or krill for treatment of psoriasis, plaques, erythrodermic psoriasis, arthropathic psoriasis, pustular psoriasis, psoriasis of the child, parapsoriasis, acute or chronic dermatoses comprising the following phospholipids, by weight relative to the total weight of phospholipids lecithin: from 10% to 75% phosphatidylcholine, preferably 45%, - from 10% to 30% of phosphatidylinositol preferably 16%, 5% to 30% of phosphatidylethanolamine, preferably 20%, 5% to 20% of phosphatidylserine, preferably 10%, and from 5% to 30% sphingomyelin, preferably 5% and said phospholipids are esterified with, by weight relative to the total weight with docosahexanoic acid at a rate of 15% to 85%, -eicosapentanoic acid at a rate of 5% at 35%, docosapentanoic acid in a proportion of 0.5% to 5%, the fatty acid of allcyl-glycerol type at a rate of 5% to 30%. However, WO2006111633 does not disclose a composition for treatment of psoriasis resulting from the addition of inositol, aspirin and autophagy activators to a pharmaceutical composition for treatment of psoriasis and diseases associated with aging and inflammatory conditions. These additions facilitating autophagy activation and treatment of root causes of inflammatory disease are not disclosed.

US2017079993 may refer to a method for treating psoriasis comprising orally administering to a subject diagnosed with psoriasis an effective amount of a lipid composition comprising phospholipids and a carrier oil, wherein the phospholipids comprise at least about 75 wt. % phosphatidylcholine; at least about 40% of the total fatty acids of the phospholipids are docosahexaenoic acid (DHA) and eicosapentaenoic acid (EPA) and combinations of said compositions with the mineral zinc; however, US2017079993 does not disclose a composition for treatment of psoriasis resulting from the addition of inositol, aspirin and autophagy activators for treatment of psoriasis and diseases associated with aging and inflammatory conditions. These additions facilitating autophagy activation and treatment of root causes of inflammatory disease are not recognized.

JP2017197472 (A) may refer to a composition for inhibiting interleukin-17A (IL-17A) production, comprising of docosahexaenoic acid (DHA) and/or eicosapentaenoic acid (EPA) as an active ingredient. However, JP2017197472 does not disclose a composition for treatment of psoriasis resulting from the addition of inositol, phosphatidylinositol, aspirin and autophagy activators to a pharmaceutical composition for treatment of psoriasis and diseases associated with aging and inflammatory conditions.

Melatonin may down regulate the expression of IL17 and positively affect treatment of psoriasis (See, Kuklina 2016). The importance of melatonin in treatment of inflammatory skin disorders such as psoriasis is supported by the observation that psoriasis flared in the winter when melatonin levels are low and cleared in summer when melatonin levels are high (Pascoe. et al. 2015; Simonneauxet al. 2003). Attributing the healing potential of melatonin in these diseases to its ability to activate autophagy and to lower levels of IL-17. Lee, B. et al. (1997) may refer to the solubility and stability of melatonin in propylene glycol and 2-hydroxypropyl-β-cyclodextrin vehicles. (see Archives of Pharmacal research, 20(6), pp.560-565) but Lee and other related references do not disclose a composition for treatment of psoriasis resulting from a combination of melatonin, herring roe oil or krill in combination with inositol, phosphatidylinositol, aspirin and autophagy activators for treatment of psoriasis and diseases associated with aging and inflammatory conditions.

Activators of peroxisome proliferator-activated receptors may not only therapeutic potential for treatment of psoriasis but also for treatment of neuroinflammatory diseases (See, Storer, 2005); pharmaceutical compositions are not known for treatment of psoriasis resulting from the combination of activators of peroxisome proliferator-activated receptors with herring roe oil, krill oil, inositol, phosphatidylinositol, aspirin and autophagy activators to a pharmaceutical composition for treatment of psoriasis and diseases associated with aging and inflammatory conditions. Thus, US2017360846 (A1) may refer to a non “WINTERIZED, STANDARDIZED MARINE SOURCE OIL PRODUCTS AND METHODS OF MAKING THEREOF” as a liquid medicament/supplement composition including oil derived from fish, krill, and/or squid, a food grade or pharmaceutically acceptable form of vitamin D3 or a derivative thereof admixed in the non-winterized marine source oil, a food grade or pharmaceutically acceptable form of vitamin A or a derivative thereof admixed in the non-winterized marine source oil, optionally a food grade or pharmaceutically acceptable form of CoQ10 or a derivative thereof admixed in the non-winterized marine source oil, a food grade or pharmaceutically acceptable form of concentrated eicosapentaenoic acid and docosahexaenoic acid or ethyl ester, glyceride ester or salt of the acid and polyphenol rich vegetable oil admixed in the non-winterized marine source oil, and optionally a food grade or pharmaceutically acceptable form of melatonin or a derivative thereof admixed in the non-winterized marine source oil. However, US2017360846 (A1) may refer to a composition containing aspirin or compositions without Vitamin D3 which is known to. upregulate the expression of the proinflammatory cytokine IL17 and hence diminish the utility of such a composition for treatment of psoriasis and inflammation associated disorders.

US2018050048 may refer to novel covalently linked binary conjugate compounds having at least one of a moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, and a moiety derived from berberine or L-carnitine or metformin, or a derivative or analog thereof and the uses of these conjugates for treating and/or preventing, for example, liver diseases or disorders, various diabetes, diabetic complications, dyslipidemia, obesity, metabolic syndromes, pre-diabetes, muscle atrophy, inflammation, and cancers; However, US2018050048, does not disclose noncovalent compositions comprising of herring roe oil, krill oil and including phosphatidylinositol or inositol.

US2015250844 may refer to covalently linked binary compositions of turmeric oil, fish oil, aspirin and anti-cancer drugs (paclitaxel) having anti-inflammatory, analgesic and/or anti-cancer activity; however, US2015250844 does not disclose noncovalent compositions including phosphatidylinositol or inositol.

US2018200216 may refer to a composition for mitigating effects of traumatic brain injury comprising docosahexaenoic acid, curcumin, and resveratrol but US2018200216 does not disclose compositions including phosphatidylinositol, inositol or aspirin.

U.S. Pat. No. 9,682,048 may refer to a multi component pharmaceutical composition for the treatment of cognitive decline including Alzheimer's disease consisting of: methylsulfonylmethane, fructose 1,6-diphosphate, and nutritional component selected from the group consisting of docosahexaenoic acid, resveratrol, and blueberry but U.S. Pat. No. 9,682,048 does not disclose a composition further including phosphatidylinositol, inositol or aspirin.

US2014308248 may refer to a Dietary Supplement System for Multifunctional Anti-Aging Management comprising (a) a telomere maintenance complex including: purslane extract (aerial parts); turmeric rhizome extract (95% curcuminoids); quercetin dehydrate, cayenne pepper fruit; vanadium (as vanadyl sulfate); fenugreek seed; astragalus root extract, omega fatty acid complex including linoleic acid; alpha-linolenic acid; oleic acid borage seed oil gamma-linolenic acid), evening primrose oil fish body oil (eicosapentaenoic acid; docosahexaenoic acid); (b) a calorie restriction mimetics and gene expression complex including trans-resveratrol (from polygonum cuspidatum root extract); pterostilbene, fisetin 50% (buxus microphylla Sieb (stem and leaf; alpha lipoic acid, coenzyme Q-10, betaine HCl, sulfur (from mthylsulfonylmethane); L-carnitine tartrate; L-carnitine HCl, and (c) a free radical scavenger complex, including green tea leaf extract catechin and polyphenols); anthocyanins (from bilberry fruit and grape skin extracts. However, US2014308248 but does not disclose a composition including phosphatidylinositol, inositol or aspirin.

US2014023701 may refer to a radioprotective mixture of micronutrients including a) a mixture of micronutrient multivitamin and trace elements, b) a mixture of antioxidants and chemo-preventative agents, and c) optionally, a mixture of fatty acids; wherein the mixture of micronutrient multivitamin and trace elements comprises: biotin, folate, iodine, manganese, pantothenic acid, selenium, vitamin B1, vitamin B12 ; vitamin B2 ; vitamin B3 ; vitamin B6 vitamin C; vitamin E, vitamin K, lycopene, zinc, alpha lipoic, astaxanthin, lutein, vitamin A, quercetin; pine bark extract; curcumin extract, calcium, chromium, copper, magnesium; molybdenum; inositol and resveratrol; however, US2014023701 does not disclose a composition containing aspirin or phosphatidylinositol.

JP2013231056 may refer to a composition for treating cellulite, preventing generation of light cellulite, preventing progress of the light cellulite to heavy cellulite, smoothening fine rise and fall of skin, maintaining or increasing a firmness property of skin, and or decreasing fat mass and surrounds in a gluteal and femoral region comprising of (−)-epigallocatechin gallate, resveratrol, eicosapentaenoic acid, docosahexaenoic acid, rosehip extract/condensate, hydroxytyrosol, lycopene, lutein, p-cryptoxanthin, zeaxanthin and a derivative thereof, however, but JP2013231056 does not disclose a pharmaceutical composition for treatment of psoriasis including aspirin, phosphatidylinositol or inositol.

WO2013152055 may refer to an oxidative exposure treatment composition comprising a mixture of micronutrient multivitamin and trace elements, a mixture of antioxidants and chemo-preventative agents, and optionally a mixture of fatty acid. Micronutrient multivitamin and trace elements mixtures include vitamins A, Bp, B1, B2, B3, B5, B6, B7, B9, B12, C, D, E and K; inositol; calcium, iodine, magnesium, zinc, selenium, copper, manganese, chromium, molybdenum, potassium, boron and vanadium. Mixtures of nonessential antioxidants and chemo-preventative agents may include bioflavins, alpha lipoic acid, N-acetyl-L-cysteine, lutein, lycopene, astaxanthin, plant sterols, isoflavones, garlic extract, which provides allicin; green tea extract, cruciferous vegetable extract, fruit extracts, ginkgo biloba extract, coenzyme Q-10, and resveratrol. Fatty acid mixtures may include eicosapentaenoic acid and docosahexaenoic acid. Methods of treatment of a subject exposed to a radiation source or an oxidative stress with the radiation—oxidative exposure treatment composition include the step of administering to the subject a daily dose of the radiation—oxidative exposure treatment composition such that the life shortening effects induced by the radiation source or the oxidative stress are ameliorated. However, WO2013152055 does not disclose a pharmaceutical composition for treatment of psoriasis including aspirin or phosphatidylinositol.

FR2221128 may refer to a composition comprising of aspirin and a natural polyol compound such as inositol for treatment of ulcers. However, FR2221128 does not disclose a composition containing eicosapentaenoic acid, docosahexaenoic acid herring roe oil or krill oil.

WO2018CA50153 may refer to a composition for treatment of traumatic brain injury comprising cannabidiol (CBD), tetrahydrocannabinol (THC), eicosapentaenoic acid and/or docosahexaenoic acid. However, WO2018CA50153 does not disclose a composition containing aspirin, phosphatidylinositol or inositol.

CN103608005 may refer to a composition of eicosapentaenoic acid and docosahexaenoic acid in combination with inositol and a chemotherapeutic agent. However, CN103608005 does not disclose a composition containing herring roe oil, krill oil, phosphatidylinositol and aspirin for treatment of psoriasis.

JPH04169524 may refer to a composition for improving serum lipid composition comprising eicosapentaenoic acid, docosahexaenoic acid and tocopherol as essential components, and further including phosphatidyl choline, phosphatidyl ethanolamine, phosphatidyl inositol, linoleic acid and gamma-linolenic acid, or oleic acid, as main components; However, JPH04169524 does not disclose a composition containing aspirin, herring roe oil, krill oil, inositol and an autophagy activator.

JPH0717855 may refer to a composition for improving cerebral function, thereby useful for the enhancement of learning ability and the prevention and treatment of senile dementia comprising docosahexaenoic acid, eicosapentaenoic acid and alpha-linolenic acid as an active ingredient and one more phospholipids selected from the phosphatidyl choline(PC), phosphatidyl ethanolamine(PE), phosphatidyl serine(PS), phosphatidyl inositol; however, JPH0717855 does not disclose a composition containing aspirin.

US2014179630 (A1) may refer to the generation of novel therapeutic agents, termed resolvins, generated from the interaction between a dietary omega-3 polyunsaturated fatty acid (PUFA) such as eicosapentaenoic acid (EPA) or docosahexaenoic acid (DHA), cyclooxygenase-II (COX-2) and aspirin and the use of these therapeutic agents for treatment of airway disease; however, US2014179630 does disclose compositions obtained from the combination of these therapeutic products with phosphatidyl inositol, inositol or autophagy activators.

US2011301239 (A1) may refer to the generation of novel therapeutic agents, termed resolvins, generated from the interaction between a dietary omega-3 polyunsaturated fatty acid (PUFA) such as eicosapentaenoic acid (EPA) or docosahexaenoic acid (DHA), cyclooxygenase-II (COX-2) and aspirin and the use of these therapeutic agents for treatment angiogenesis; however, US2011301239 (A1) does disclose compositions obtained from the combination of these therapeutic products with phosphatidyl inositol, inositol or autophagy activators.

WO2006078457 may refer to the generation of novel therapeutic agents, termed resolvins, generated from the interaction between a dietary omega-3 polyunsaturated fatty acid (PUFA) such as eicosapentaenoic acid (EPA) or docosahexaenoic acid (DHA), cyclooxygenase-II (COX-2) and aspirin and the use of these therapeutic agents for treatment gastrointestinal diseases angiogenesis; however, WO2006078457 does disclose compositions obtained from the combination of these therapeutic products with phosphatidyl inositol, inositol or autophagy activators.

Song, W. et al. (2014) may refer to that 2-Hydroxypropyl-β-cyclodextrin promotes TFEB-mediated activation of autophagy: Implications for therapy. See, Journal of Biological Chemistry, pp.jbc-M113. However, Song does not disclose compositions for treatment of psoriasis obtained from the combination of herring roe or krill oil in combinations with phosphatidyl inositol, inositol and autophagy activators.

Ory D. S. et al. (2017) may refer to Intrathecal 2-hydroxypropyl-β-cyclodextrin decreases neurological disease progression in Niemann-Pick disease, type C1: a non-randomized, open-label, phase 1-2 trial. The Lancet, 390(10104), pp.1758-1768. However, Ory does not disclose compositions for treatment of psoriasis obtained from the combination of Herring roe or krill oil in combinations with phosphatidyl inositol, inositol and autophagy activators.

Acosta, E. Herrera. et al. (2016) may refer to a composition of an olive polyphenolic extract, retinoic acid, vitamin A, riboflavin, that improves cutaneous manifestations of psoriasis in humans; However, Acosta does not disclose compositions for treatment of psoriasis obtained from the combination of Herring roe or krill oil in combinations with phosphatidyl inositol, inositol and autophagy activators.

U.S. Pat. No. 6,440,465 may refer to compositions and methods of use thereof for the treatment of psoriasis and related skin comprising glucosamine, berberine and oleuropein; however, U.S. Pat. No. 6446465does not disclose compositions for treatment of psoriasis obtained from the combination of Herring roe or krill oil in combinations with phosphatidyl inositol, inositol and autophagy activators.

Park, G. et al. (2016) may refer to that 6-Shogaol, an active compound of ginger, alleviating allergic dermatitis-like skin lesions via cytokine inhibition by activating the Nrf2 pathway; however, Park et al. does not disclose compositions for treatment of psoriasis obtained from the combination of Herring roe or krill oil in combinations with phosphatidyl inositol, inositol and autophagy activators.

Singh, S. et al. (2016) may refer to Metformin and Pioglitazone being used to treat psoriasis in patients; however, Sing does not diclsose compositions for treatment of psoriasis obtained from the combination of Herring roe or krill oil in combinations with phosphatidylinositol and inositol

US2017209397 may refer to a composition comprising β-hydroxy-β-methylbutyrate (HMB) and methods of using HMB to treat, prevent or improve diseases or conditions that can benefit from autophagy enhancement and/or modulation; however, US2017209397 does not disclose compositions comprising p-hydroxy-β-methylbutyrate in combination Herring roe or krill oil and phosphatidylinositol or inositol and aspirin for treatment of psoriasis.

WO2018148113 may refer to pharmaceutical compositions comprising isotope enhanced ambroxol, alone or in combination with an additional bioactive agent, especially rifamycin antibiotics, including an additional autophagy modulator (an agent which is active to promote or inhibit autophagy) for treatment of autophagy mediated disease state and/or condition), especially Mycobacterium infection. Chronic Obstructive Pulmonary Disease (COPD), asthma, pulmonary fibrosis, cystic fibrosis, Sjogren's disease and lung cancer (small cell and non-small cell lung cancer; however, WO2018148113 does not disclose compositions comprising ambroxol in combination Herring roe or krill oil and phosphatidylinositol or inositol and aspirin for treatment of psoriasis.

US20080089876A1 may refer to a pharmaceutical composition consisting essentially of cis 5,8,11,14.17-eicosapentanoic acid and cis 4,7,10,13,16,19-docosahexanoic acid(DHA), one of their esters or pharmaceutically accept able salts or mixture thereof, a statin, Coenzyme Q10, resveratrol, a policosanol, pantethine, Selenium and

Zinc, and the use of this composition for treatment of diabetes; however, US20080089876A1 does not disclose a composition containing combinations of omega3 fatty acids in combination with aspirin, phosphatidylinositol and autophagy activators or inducers for treatment of inflammatory disorders.

Matsunaga S. et al. (2015) may refer to results of clinical trials using Lithium as a Treatment for Alzheimer's Disease and report that lithium slows cognitive declines in patients with Alzheimer's disease.

US2013011468 may refer to the use of a fatty acid composition comprising of docosahexaenoic acid (DHA), or derivatives thereof, and eicosapentaenoic acid (EPA), or derivatives thereof for the treatment and/or prevention of amyloidos-related diseases, such as Alzheimer's disease, as well as treatment/prevention of cognitive dysfunction; US2013011468 does not disclose a composition containing combinations of omega3 fatty acids in combination with aspirin, phosphatidylinositol and autophagy activators or inducers for the treatment and/or prevention of amyloidos-related diseases, such as Alzheimer's disease, as well as treatment/prevention of cognitive dysfunction or dermatological and cardiovascular disease.

EP2140863 may refer to the use of docosahexaenoic acid (DHA) for the manufacture of a medicament for the treatment of neurological disorders including Alzheimer's disease Zellweger's syndrome, neonatal adrenoleukodystrophy, infantile Refsum disease, hyperpepecolic acidemia, Rhizomelic chondrodysplasia punctata, Zellweger-like syndrome, adrenoleukodystrophy, adrenomyeloneuropathy, acyl-CoA oxidase deficiency, bifunctional protein deficiency, thiolase deficiency, hyperoxaluria type I, acatalasaemia adult Refsum disease, diabetic neuropathy, schizophrenia and Huntington's disease and senile dementia; however, EP2140863 describe a composition containing combinations of DHA in combination with aspirin, phosphatidylinositol and autophagy activators for the treatment and/or prevention of these diseases.

U.S. Pat. No. 5,288,755 may refer to physiologically active and nutritional composition consisting of linoleic acids of linoleic acid and at least one member of the group consisting of C-8-18 saturated fatty acids, oleic acid and derivatives of these acids for supporting memory enhancement, analgesia, sleep regulation and inhibition of the symptoms of senility; however, U.S. Pat. No. 5,288,755 does not disclose a composition containing combinations of linoleic acids in combination with aspirin, phosphatidylinositol and autophagy activators for the treatment and/or prevention of these diseases or conditions.

US2004039058 may refer to compositions containing eicosapentaenoic acid and or stearidonic acid for treatment and prevention of inflammatory disorders; however, US2004039058 does not disclose compositions of these fatty acids in combination with aspirin, phosphatidylinositol and autophagy activators for the treatment and/or prevention of these diseases or conditions.

U.S. Pat. No. 5,599,840 (A) may refer to a physiologically active and nutritional composition comprising of at least one member of the group consisting of C8-18 saturated fatty acids, oleic acid and derivatives of these acids, (2) a composition of matter which consists of (a) from about 13.0 to about 27.5% by weight of at least one compound selected from the group consisting of linolenic acid and salts, esters, and amides thereof; however, U.S. Pat. No. 5,599,840 does not disclose a composition containing combinations of these fatty acids with aspirin, phosphatidylinositol and autophagy activators.

U.S. Pat. No. 5,198,468 (A), U.S. Pat. No. 5,120,763 and EP0366480 may refer to an essential fatty acid composition for improving memory deterioration associated with schizophrenia or physiological aging using essential fatty acid including EPA and DHA in an effective amounts of 1 mg to 100 g per day; however, U.S. Pat. No. 5,198,468 (A), U.S. Pat. No. 5,120,763 and EP0366480 do not disclose a composition containing combinations of linoleic acids in combination with aspirin, phosphatidylinositol and autophagy activators for the treatment and/or prevention of these diseases or conditions.

US2007161705 may refer to a composition including alpha linoleic acid, docosahexaenoic acid and eicosapentaenoic acid for treatment of epilepsy, schizophrenia, bipolar (manic-depressive illness) and unipolar (major depression) psychiatric disorders, degenerative Alzheimer's disease and related forms of dementia. schizophrenia or physiological aging using essential fatty acid including EPA and DHA; however, US2007171705 does not disclose a composition containing combinations of these fatty acids in combination with aspirin, phosphatidylinositol and autophagy activators for the treatment and/or prevention of these diseases or conditions.

U.S. Pat. No. 5,120,760 may refer to the use of essential fatty acid compositions containing EPA and DHA for treating tardive dyskinesia; however, U.S. Pat. No. 5,120,760 does not disclose a composition containing combinations of linoleic acids in combination with aspirin, phosphatidylinositol, 2-hydroxypropylcyclodextrine and autophagy activators or inducers for the treatment and/or prevention of these diseases or conditions.

US2006166935 (A1) may refer to a fatty acid composition for treatment of alzheimer's disease and cognitive dysfunction consisting of docosahexaenoic acid (DHA), or derivatives thereof, and (all-Z omega-3)-5,8,11,14,17-eicosapentaenoic acid (EPA), or derivatives thereof; however, US2006166935 does not disclose a composition containing combinations of linoleic acids in combination with aspirin, phosphatidylinositol, 2-hydroxypropylcyclodextrine and autophagy activators or inducers for the treatment and/or prevention of these diseases or conditions.

EP0707487 may refer to use of an oil comprising triglycerides and/or phospholipids containing docosahexanoic acid (DHA) for the preparation of a composition for the treatment of a neurological disorder associated with a DHA deficiency-associated pathology, wherein said oil is fish oil and said disorders are retinitis pigmentosum, diabetic neuropathy and multiple sclerosis; however, EP0707487 does not disclose a composition containing fish oil constituents in combination with aspirin, phosphatidylinositol, 2-hydroxypropylcyclodextrine and autophagy activators or inducers for the treatment and/or prevention of these diseases or conditions.

EP0347056 may refer to compositions containing EPA and DHA for treatment of inflammatory disorder including atopic eczema, psoriasis, acne, contact dermatitis or urticaria; however, EP0347056 does not disclose a composition containing fish oil constituents in combination with aspirin, phosphatidylinositol, 2-hydroxypropylcyclodextrine and autophagy activators or inducers for the treatment and/or prevention of these diseases or conditions.

EP0342795 (A2) may refer to the use of docosahexaenic acid and foods containing DHA for improving brain function; however, EP0342795 does not disclose a composition containing fish oil constituents in combination with aspirin, phosphatidylinositol, 2-hydroxypropylcyclodextrine and autophagy activators or inducers for the treatment and/or prevention of these diseases or conditions

EP0296751 may refer to a medicament for treatment or prevention of memory loss, including EPA and DHA; however, EP0296751 does not disclose a composition containing fish oil constituents in combination with aspirin, phosphatidylinositol, 2-hydroxypropylcyclodextrine and autophagy activators or inducers for the treatment and/or prevention of these diseases or conditions.

EP1419780 (A1) may refer to the use of docosahexaenoic acid (DHA) for the manufacture of a medicament for the treatment of senile dementia and Alzheimer's disease; however, EP1419780 does not disclose a composition containing DHA in combination with aspirin, phosphatidylinositol, 2-hydroxypropylcyclodextrine and autophagy activators or inducers for the treatment and/or prevention of these diseases or conditions.

US2016095888 (A1) may refer to compositions and methods using krill oil to treat risk factors for metabolic, cardiovascular, and inflammatory disorders and also relates to methods of using these compositions for modulating biological processes selected from the group consisting of glucose metabolism, lipid biosynthesis, fatty acid metabolism, cholesterol biosynthesis, and the mitochondria respiratory chain; however, US2016095888 does not disclose a composition containing krill oil in combination with aspirin, phosphatidylinositol, 2-hydroxypropylcyclodextrine and autophagy activators for the treatment and/or prevention of these diseases or conditions.

EP2153736 may refer to dietary composition for inhibiting the activity of a leukotriene receptor, comprising a phospholipid extract of a marine product; however, EP2153736 does not disclose a composition containing krill oil in combination with aspirin, phosphatidylinositol, 2-hydroxypropylcyclodextrine and autophagy activators for the treatment and/or prevention of these diseases or conditions.

US2015306132 and US2014005267 may refer to a composition and method to treat and alleviate symptoms of joint pain comprising phospholipids and at least one of polyunsaturated EPA and DHA in combination with or without astaxanthin and pro-inflammatory low molecular weight microbial fermented sodium hyaluronate fragments; however, US2015306132 and US2014005267 do not disclose a composition containing krill oil in combination with aspirin, phosphatidylinositol, 2-hydroxypropylcyclodextrine and autophagy activators for the treatment and/or prevention of these diseases or conditions.

WO2015181640 may refer to krill oil compositions for treating cardiovascular disease (CVD), treating cognitive disease, treating inflammatory disease, treating obesity, treating abdominal obesity, preventing CVD, preventing cognitive disease, preventing inflammatory disease, preventing obesity, preventing abdominal obesity, reducing high blood pressure, reducing heart rate, reducing blood plasma concentrations of saturated fatty acids, reducing blood plasma concentrations of palmitic acid, increasing blood plasma concentrations of Alpha-Linolenic acid (ALA) and increasing blood plasma concentrations of stearic acid; however, WO2015181640 does not disclose a composition containing krill oil in combination with aspirin, phosphatidylinositol, 2-hydroxypropylcyclodextrine and autophagy activators for the treatment and/or prevention of these diseases or conditions.

US2015182561 and WO2015142702 may refer to dietary supplement compositions formulated in a therapeutic amount to treat and alleviate symptoms of joint pain comprising astaxanthin, low molecular weight hyaluronic acid or sodium hyaluronate (hyaluronan) and a phospholipid rich egg roe extract having phospholipid bound EPA and DHA admixed with seed and/or fish oil having an ALA to LA ratio of 1:1 to 6:1 and in an oral dosage form; however, US2015182561 and WO2015142702 do not disclose a composition containing egg roe extracts containing EPA and DHA in combination with aspirin, phosphatidylinositol, 2-hydroxypropylcyclodextrine and autophagy activators for the treatment and/or prevention of these diseases or conditions.

U.S. Pat. No. 5,436,269 may refer to a composition containing docosahexaenoic acid (DHA) or a derivative thereof as an active ingredient for reducing triglyceride levels and treating fatty liver, hepatic cirrhosis, chronic hepatitis, hepatocirrhosis, hepatocellular carcinoma and hepatic insufficiency; however, U.S. Pat. No. 5,436,269 does not disclose a composition containing DHA in combination with aspirin, phosphatidylinositol, 2-hydroxypropylcyclodextrine and autophagy activators for the treatment and/or prevention of these diseases or conditions.

WO2005063231 may refer to a composition containing docosahexaenoic acid (DHA), linoleic acids and eicospentaenoic acids or a derivative thereof in combination with a triterpene or an ester thereof for treatment of physiological and disease states selected from the group comprising rheumatoid arthritis, osteoarthritis, back-ache, psoriasis, pre-menstrual syndrome, bacterial infections, viral infections, fatigue, insomnia, anxiety, obesity, influenza, diabetes mellitus, alcoholism, cancer, neurological disorders, epilepsy, tardive dyskinesia and choreiform disorders, psychiatric disorders, cardiovascular disorders, dermatological disorders, respiratory disorders, learning disabilities and ageing; however, WO2005063231, does not disclose compositions containing EPA and DHA, linoleic acid triterpene or their derivatives in combination with aspirin, phosphatidylinositol, 2-hydroxypropylcyclodextrine and autophagy activators for the treatment and/or prevention of these diseases or conditions.

EP0457950 and EP1489915 may refer to compositions including stearidonic acid, docosahexaenoic acid and eicosapentaenoic acid for the treatment of inflammatory disorders; however, EP0457950 and EP1489915 does not disclose compositions containing EPA and DHA, stearidonic acid or their derivatives in combination with aspirin, phosphatidylinositol, 2-hydroxypropylcyclodextrine and autophagy activators for the treatment and/or prevention of these diseases or conditions.

WO9848788 (A1) may refer to compositions including docosahexaenoic acid (DHA), linoleic acids and eicospentaenoic acids (EPA) for treatment of depression, anxiety; however, WO9848788 does not disclose compositions containing EPA and DHA, linoleic acid triterpene or their derivatives in combination with aspirin, phosphatidylinositol, 2-hydroxypropylcyclodextrine and autophagy activators for the treatment and/or prevention of these diseases or conditions.

WO0044360 may refer to fatty acid compositions including DHA, EPA and linoleic acid susceptible to metabolism by FACL enzymes and particularly by FACL-4 to Coenzyme A derivatives for treatment of schizophrenia, schizo-affective disorders, schizotypal disorder, mania, depression, bipolar disorder, ADHD, anxiety and panic attacks, social phobias, Alzheimer's disease and other dementias, epilepsy, Parkinson's disease, stroke, transient ischaemic attack, multiple sclerosis, Huntington's disease and other neurodegenerative disorders; however, WO0044360 does not disclose compositions containing EPA and DHA, linoleic acid in combination with aspirin, phosphatidylinositol, 2-hydroxypropylcyclodextrine and autophagy activators for the treatment and/or prevention of these diseases or conditions.

FR2833174 (A1) may refer to fish-oil compositions for treatment of dermatitis, atopic eczema and psoriasis, skin dryness and hyperkeratinosis; however, FR2833174 does not disclose compositions containing fish oil in combination with aspirin, phosphatidylinositol, 2-hydroxypropylcyclodextrine and autophagy activators for the treatment and/or prevention of these diseases or conditions.

WO2014143275 may refer to compositions including of eicosapetaenoic acid (EPA) docosahexaenoic acid (DHA) and docosapentaenoic acid (DPA) for treatment of fatty liver, non-alcoholic steatohepatitis (NASH), non-alcoholic fatty liver disease (NAFLD), alcoholic steatohepatitis (ASH), hepatitis, HIV (human immunodeficiency virus) infection, drug-induced fatty liver or sequelae, liver failure, liver transplantation, transplanted liver failure, liver damage associated with renal failure or disease, abnormally elevated liver enzymes, or type 2 diabetes; however, WO2014143275 does not disclose a composition containing EPA, DHA and DPA in combination with aspirin, phosphatidylinositol, 2-hydroxypropylcyclodextrine and autophagy activators for the treatment and/or prevention of these diseases or conditions.

US2014100273 may refer to compositions consisting of Docosapentaenoic acid, Eicosapentaenoic acid and Docosahexaenoic acid for reducing lipid parameters, such as triglycerides, total cholesterol, low density lipoprotein (LDL) cholesterol, non-HDL cholesterol, free fatty acids, and other lipids; however, US2014100273 does not disclose a composition containing EPA, DHA and DPA in combination with aspirin, phosphatidylinositol and autophagy activators for the treatment and/or prevention of these diseases or conditions.

WO2017158355 (A1) may refer to compositions for treatment of obesity including docosapentaenoic acid, eicosapentaenoic acid and docosahexaenoic acid, however, WO2017158355 does not disclose compositions containing EPA, DHA and DPA in combination with aspirin, phosphatidylinositol, 2-hydroxypropylcyclodextrine and autophagy activators.

WO2011072132 (A1) may refer to compositions including docosapentaenoic acid, and eicosapentaenoic acid for treating inflammatory condition associated with P-arrestin2 function, such as diabetes and obesity; however, WO2011072132 does not disclose compositons containing EPA, DHA in combination with aspirin, phosphatidylinositol, 2-hydroxypropylcyclodextrine and autophagy activators.

Zimmer S. et al, (2016) may refer to the use of cyclodextrins including 2 -hydroxyproply cyclodextrin as Emerging Therapeutic Tools in the Treatment of Cholesterol-Associated Vascular and Neurodegenerative Diseases.

Crini, G. et al. (2018) may refer to applications of Cyclodextrins. Cyclodextrin Fundamentals, Reactivity and Analysis, pp.1-55.

EP0234733 may refer to the use of essential fatty acid or lithium salt thereof and combinations with lithium acetylsalicylate for treatment of dementia wherein said fatty acids are selected from the group consisting of dihomo gamma-linolenic acid, arachidonic acid, eicosapentaenoic acid, docosahexaenoic acid, linoleic acid, gamma linolenic acid, alpha linolenic acid, 18:4n-3; however, EP0234733 does not disclose the use of lithium acetyl salicylate in combination with krill oil or herring roe oil and does not describe the use of lithium salts of dihomo gamma-linolenic acid, arachidonic acid, eicosapentaenoic acid, docosahexaenoic acid, linoleic acid, gamma linolenic acid, alpha linolenic acid, 18:4n-3 in combination with inclusion complexes of aspirin with 2-hydroxypropyl cyclodextrin.

The aspects of the present disclosure relate to novel pharmaceutical compositions and methods of use thereof, the pharmaceutical compositions including herring roe oil, krill oil, fish oils, eicosapentaenoic acid, docosahexaenoic acid, stearidonic acid, Phosphatidylinositol and inositol derived from chemical synthesis or natural sources such as fish, algae's and fruits and further comprising of substances capable of increasing COX 2 mediated metabolisms of eicosapentaenoic acid and docosahexaenoic acid selected from the group consisting of aspirin (See, Serhan C. N. 2002) and further comprising of substances capable of reducing SQSTM1 levels by activating autophagy selected from the group consisting of spermidine (See, Morselli, E. et al. 2011), melatonin (See, Hong Y. et al. 2014), 2-hydroxypropyl-β-cyclodextrin (See, Barbero-Camps E. et al. 2018), beta-hydroxy-beta-methylbutyrate (See, Abumrad Najinet al., 2017), hydroxytyrosol (See, Cetrullo S. et al. 2016), 6-shogaol (See, Hung J. Y. et al. 2009), resveratrol (See, Kumar B. et al. 2015), pterostilbene (See, Chen, R. J.et al. 2010), vitis vinifera, quercetin (See, Russo, M., et al. 2012.), various zinc salts (See, Hung H. H. et al. 2013), retinoic acid (See, Rajawat Y. 2010), berberin (See, Fan X., et al. 2015), estradiol (See, Yang Y. H. et al. 2013), cannabidiol (See, Yang L. et al. 2014), cannabinoids (See, Dando I. et al. (2013), HU-308 (See, Shao B. Z. 2014), curcumin See, Yang, C., et al. 2017), acetyl-11-keto-boswellic acid (See, Al Zadjali, F. et al. 2018), glucosamine (See, Carames, B. et al. 2013), epigallocatechin-3-gallate (See, Zhou J. et al. 2014), oleuropein aglycone (See, Rigacci S. et al. 2015), metformin (See, Song Y. M. et al. 2015), fasudil (See, Gao, H.et al. 2016), ripasudil (See, Kitaoka Y. et al. (2017), lanthionine ketimine, lanthionine ketimine-5-ethyl ester (See, U.S. Patent 7,683,055), ambroxol (See, Choi S. W. et al. 2018), isorhamnetin, genkwanin, acacetin (See, Zhang, H. W. et al. 2018), diclofenac, acetohexamide, fulvestrant, astaxanthin, fusaric acid, GW7647, LY-307265, indatralin HCl, CAPE, SMER-28, grayanotoxin III, vitexin, doxycycline, neomycin, lithium chloride, nimodipine, N5-methyl adenosine, ritodrine, AB-MECA, esomeprazole, SB242084, rottlerin (See, Piyush M. 2017), telmisartan (See, Li, B. H.et al. 2015) urolithin A, urolithin B, urolithin C (See, Zhao, W., et al. 2018), perifosine (See, Tong Y.et al, 2012), perhexiline, niclosamide, amiodarone (See, Balgi A. D. et al. 2009), rilmenidine (See, Lin F. and Qin Z. H. 2013), clonidin, fluspirilene, trifluoperazine, pimozide, niguldipine, loperamide, nitrendipine, penitrem A (See, Renna, M., Jet al. 2010), minoxidil (See, Williams, A.et al. 2008), NF449 (See, Menzies, F. M., et al. 2015), valproate, trehalose, carbamazepine (See, Sarkar S. et al. 2008), pioglitazone (See, Hsiao, et al. 2017), rosiglitazone (See, Cerquetti L.et al. 2011), caffeine (See, Moon J. H. et al. 2014), diosgenin (See, Zhang B. et al. 2017), atorvastatin (See, Zhang, Q. et al. 2012), Genistein (See, Wang Yet al. 2018), dauricine, cepharanthine (See, Law B. Y. 2014), thalidezine (See, Law B. Y. 2017).

The pharmaceutical compositions and combinations of the present disclosure are useful for treatment and methods of treatment of diseases in a mammal selected from the group consisting of psoriasis, arthritic psoriasis, pediatric psoriasis, plaques, acne, atopic dermatitis, depressive disorders, multiple sclerosis, bone loss, colitis, inflammatory bowel disease, Crohn's Disease, ulcer, rheumatoid arthritis, cardiovascular disease (e.g., atherosclerosis, cardiomyopathy, congestive heart failure, endocarditis), renal disease, liver diseases (e.g., nonalcoholic fatty liver disease, nonalcoholic steatohepatitis, and primary biliary cholangitis), lung diseases (e.g., interstitial lung disease, idiopathic pulmonary fibrosis, hypersensitivity pneumonitis, sarcoidosis, and asbestosis), aging associated diseases (e.g., atherosclerosis, arthritis, cataracts, osteoporosis, type 2 diabetes, hypertension, Alzheimer's disease, hearing loss, maculopathy, osteoarthritis, Parkinson's disease, periodontitis, rheumatoid arthritis, and sarcopenia), post-traumatic stress disorder, dyspraxia, autism spectrum disorders, stroke, Rett syndrome, Parkinson's disease, Alzheimer' s disease, diabetes, obesity, infertility, traumatic brain injury, skin disorders and various diseases caused by the hyperproliferation of cells such as cancers (e.g., colorectal cancer, osteomyelitis, Hodgkin Disease, Bladder cancer, prostate cancer, ovarian cancer, gall bladder cancer, pancreatic cancer, esophageal cancer, liver cancer, mesothelioma and lung cancer). Said pharmaceutical compositions of the present disclosure will also have utility for reducing pain and inflammation by reducing the expression and protein levels of inflammation associated proteins selected from the group consisting of COX 2, VEGF2, IL23, 1117, IL1, IL6, TNF alpha by increasing the production of anti-inflammatory resolvins, protectins, marensis and by increasing the lysosomal degradation of SQSTM1 .

The novel pharmaceutical compositions, combinations and methods of treatment embodiments of the present disclosure can prevent or ameliorate psoriasis characterized by epidermal thickening and T cell invasion increased vascularization and increased secretion of inflammatory cytokines.

A pharmaceutical composition and embodiments thereof of the present disclosure can be directly administered as the preventing or ameliorating agent for skin psoriasis of the present disclosure. However, it is desirable to provide the agent in the form of a variety of drug formulations. Such drug formulation contains combinations of active ingredients. Further, the drug formulation can be produced by an arbitrary method that has been well known in the art of drug formulation by mixing the active ingredient with at least one type of pharmacologically acceptable carrier or vehicle. It is desirable that the most effective administration route of drug formulation would be selected for treatment. Examples thereof include oral administration, topical administration and parenteral administration such as intravenous, intraperitoneal, or subcutaneous administration. However, oral administration is preferable. Examples of dosage forms that can be used for administration include: oral agents such as tablets, powders, granules, pills, Suspensions, emulsions, infusions and decoctions, capsules, syrups, liquid, elixirs, extracts, tinctures, and fluid extracts; and parenteral agents such as parenteral injections, intravenous fluids, creams, and Suppositories. The composition in the form of an oral composition is preferably used.

In the case involving the use of liquid preparations such as syrup appropriate for oral administration, the preparations can be formulated by addition of water, Sugars such as Sucrose, Sorbitol, and fructose; glycols such as polyethylene glycol and propylene glycol, oils such as Sesame oil, olive oil, and soybean oil; antiseptics such as p-hydroxy benzoate esters; parahydroxy benzoate derivatives such as methyl parahydroxy benzoate; preservatives such as Sodium benzoate; and flavors such as Strawberry flavor and peppermint flavor.

In addition, in the case involving the use of tablets, powders, and granules that are appropriate for oral administration the preparations can be formulated by addition of sugar such as lactose, glucose, sucrose, mannitol, and sorbitol; starch from potatoes, wheat, and corn; an inorganic substance such as calcium carbonate, calcium sulfate, sodium bicarbonate, and sodium chloride; an excipient of a plant-derived powder such as crystalline cellulose, a sweetroot powder and gentian powder, a disintegrator Such as starch, agar, gelatin powder, crystalline cellulose, carmellose sodium, carmellose calcium, calcium carbonate, sodium bicarbonate, and sodium alginate; a lubricant such as magnesium stearate, talc, hydrogenated plant oil, macrogol, and silicone oil; a binder such as polyvinyl alcohol, hydroxypropyl cellulose, methyl cellulose, ethylcellulose, carmellose, gelatin, and starch paste liquid; a surfactant such as fatty acid ester, and a plasticizer such as glycerine.

It is also possible to add an additive generally used for foods and beverages to the drug formulation appropriate for oral administration. Examples of additives include sweeteners, colorants, preservatives, thickening stabilizers, antioxidants, coloring agents, bleaches, antifungal agents, gumbases, bittering agents, enzymes, gloss agents, acidulants, seasonings, emulsifiers, fortifiers, production agents, aroma chemicals, and spice extracts.

The pharmaceutical composition formulation embodiments of the present disclosure appropriate for oral administration may be directly used in the form of, for example, a powder food product, a sheet-type food product, a bottled food product, a canned food product, a retort food product, a capsule food product, a tablet food product, a liquid food product, or a drink. In addition, the drug formulation may be used in the form of food or beverage such as health food, functional food, nutritional supplement, or food for specified health use for prevention and amelioration of skin psoriasis. For example, a parenteral injection appropriate for parenteral administration comprises preferably a sterilized aqueous agent which contains a composition of the present disclosure and which is isotonic to the blood of a recipient. For example, for a parenteral injection, an injectable solution is prepared with the use of a pharmaceutically acceptable carrier or vehicle comprising a salt solution, a glucose solution, or a mixture of a salt Solution and a glucose solution.

In addition, it is also possible to add at least one supplemental component to a parenteral agent, wherein Such components can be selected from the group consisting of diluents, antiseptics, flavors, excipients, disintegrators, lubricants, binders, Surfactants, and plasticizers, which are described above for an oral agent.

The concentration of a pharmaceutical composition, combination and methods of treatment embodiments as well as the different components incorporated into the pharmaceutical composition and combination embodiments of the present disclosure in the preventing or ameliorating agent for skin psoriasis utilizing embodiments of the present disclosure is adequately determined depending on the type of drug formulation, effects expected to be obtained because of administration of the drug formulation, and the like.

For instance, in the case of an oral agent, the concentration of a pharmaceutical composition embodiments or the components thereof of the present disclosure or a salt thereof is generally 0.1 to 100% by weight.

The dosage and the number of doses of the preventing or ameliorating agent for skin psoriasis of the present disclosure would vary depending on dosage form, patients ages and body weights, characteristics of symptoms to be treated, and severity of a symptom. However, for administration, the dosage in terms of a composition of the present disclosure or a salt thereof for an adult per day is generally 100 mg to 10000 mg once daily or separately over several times a day.

The dosing period is not particularly limited. However, it is generally 1 day to 1 year.

In addition, the drug formulations, pharmaceutical compositions, combinations and method of treatment embodiments of the present disclosure can be used not only for humans but also for animals.

Examples of animals include mammals, birds, reptiles, amphibians, and fish.

When the preventing or ameliorating agent for skin psoriasis involving embodiments of the present disclosure are administered to an animal, the dosage and the number of doses would vary depending on dosage form, and animal age and type. However, the agent is administered in the form of embodiments of the present disclosure in a dose of generally 50 mg to 10000 mg depending on body weight once daily or separately over several times a day. The dosing period is in general 1 day to 1 year.

An embodiment of the present disclosure includes a pharmaceutical composition or combination comprising of at least one ingredient selected from a first group of ingredients consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil further comprising of at least one of a second group of ingredients consisting of phosphatidylinositol, inositol, and aspirin; at least one of a third group of ingredients consisting of 2-hydroxypropyl-β-cyclodextrin, beta-hydroxy-beta-methylbutyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus; and a pharmaceutically acceptable carrier.

An embodiment of the present disclosure includes a pharmaceutical composition or combination comprising of about 100 mg to about 10000 mg Herring roe oil further comprising of phosphatidylinositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising of about 100 mg to about 10000 mg of eicosapentaenoic acid further comprising of phosphatidylinositol in amounts of about 0.8-about 50% of the weight of the total phospholipid composition further comprising of about 82 mg to about 800 or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising of about 100 mg to about 10000 mg docosahexaenoic acid further comprising of phosphatidylinositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising of about 100 mg to about 10000 mg linoleic acids further comprising of phosphatidylinositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising of about 100 mg to about 10000 mg krill oil further comprising of phosphatidylinositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising of about 100 mg to about 10000 mg Krill oil further comprising of inositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising about 100 mg to about 10000 mg Herring roe oil, further comprising of inositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising about 100 mg to about 10000 mg eicosapentaenoic acid further comprising of inositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising about 100 mg to about 10000 mg Herring roe oil and about 82 mg to about 800 mg or about 82 mg to about 200 mg aspirin in vehicles containing about 300- about 1200 mg 2-hydroxypropyl-β-cyclodextrin as active principles in association with one or more pharmaceutically acceptable adjuvants and/or excipients and preferably 2-hydroxypropyl-β-cyclodextrin.

An embodiment of the present disclosure includes a pharmaceutical composition or combination comprising about 100 mg to about 10000 mg krill oil and about 82 mg to about 800 mg or about 82 mg to about 200 mg aspirin in vehicles containing about 300- about 1200 mg 2-hydroxypropyl-3-cyclodextrin as active principles in association with one or more pharmaceutically acceptable adjuvants and/or excipients.

An embodiment of the present disclosure includes a pharmaceutical composition or combination comprising about 100 mg to about 10000 mg eicosapentaenoic acid and about 82 mg to about 800 mg or about 82 mg to about 200 mg aspirin in vehicles containing about 300- about 1200 mg 2-hydroxypropyl-3-cyclodextrin as active principles in association with one or more pharmaceutically acceptable adjuvants.

An embodiment of the present disclosure includes a pharmaceutical composition or combination comprising about 100 mg to about 10000 mg Herring roe oil and about 82 mg to about 800 mg or about 82 mg to about 200 mg aspirin and about 5 mg to about 200 mg melatonin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg krill oil and about 82 mg to about 800 mg or about 82 mg to about 200 mg aspirin and about 5 mg to about 200 mg melatonin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg eicosapentaenoic acid and about 82 mg to about 800 mg or about 82 mg to about 200 mg aspirin and about 5 mg to about 200 mg melatonin in vehicles containing about 300- about 1200 mg 2-hydroxypropyl-3-cyclodextrin as active principles in association with one or more pharmaceutically acceptable adjuvants and/or excipients.

An embodiment of the present disclosure includes a pharmaceutical composition or combination comprising about 100 mg to about 10000 mg docosahexaenoic acid and about 82 mg to about 800 mg or about 82 mg to about 200 mg aspirin and about 5 mg to about 200 mg melatonin in vehicles containing about 300- about 1200 mg 2-hydroxypropyl-3-cyclodextrin as active principles in association with one or more pharmaceutically acceptable adjuvants and/or excipients.

An embodiment of the present disclosure includes a pharmaceutical composition or combination comprising about 100 mg to about 10000 mg linoleic acids and about 82 mg to about 800 mg or about 82 mg to about 200 mg aspirin and about 5 mg to about 200 mg melatonin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg herring roe oil about 82 mg to about 800 mg or about 82 mg to about 200 mg aspirin and about 0.5 to about 10000 mg metformin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg krill oil about 82 mg to about 800 mg or about 82 mg to about 200 mg aspirin and about 0.5 to about 10000 mg metformin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg eicosapentaenoic acid about 82 mg to about 800 mg or about 82 mg to about 200 mg aspirin and about 0.5 to about 10000 mg metformin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg herring roe oil about 82 mg to about 800 mg or about 82 mg to about 200 mg aspirin and about 0.5 to about 40 mg of a statin selected from the group consisting of simvastatin, lovastatin, fluvastatin, pravastatin, atorvastatin, cerivistatin, Monascus purpureus, rosuvastatin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg krill oil about 82 mg to about 800 mg or about 82 mg to about 200 mg aspirin and about 0.5 to about 40 mg of a statin selected from the group consisting of simvastatin, lovastatin, fluvastatin, pravastatin, atorvastatin, cerivistatin, Monascus purpureus, rosuvastatin in vehicles containing about 300- about 1200 mg 2-hydroxypropyl-3-cyclodextrin as active principles in association with one or more pharmaceutically acceptable adjuvants and/or excipients.

An embodiment of the present disclosure includes a pharmaceutical composition or combination comprising about 100 mg to about 10000 mg eicosapentaenoic acid and about 82 mg to about 800 mg or about 82 mg to about 200 mg aspirin and about 0.5 to about 40 mg of a statin selected from the group consisting of simvastatin, lovastatin, fluvastatin, pravastatin, atorvastatin, cerivistatin, Monascus purpureus, rosuvastatin in vehicles containing about 300- about 1200 mg 2-hydroxypropyl-β-cyclodextrin as active principles in association with one or more pharmaceutically acceptable adjuvants and/or excipients.

An embodiment of the present disclosure includes a pharmaceutical composition or combination comprising about 100 mg to about 10000 mg docosahexaenoic acid and about 82 mg to about 800 mg or about 82 mg to about 200 mg aspirin and about 0.5 to about 40 mg of a statin selected from the group consisting of simvastatin, lovastatin, fluvastatin, pravastatin, atorvastatin, cerivistatin, Monascus purpureus, rosuvastatin in vehicles containing about 300- about 1200 mg 2-hydroxypropyl-13-cyclodextrin as active principles in association with one or more pharmaceutically acceptable adjuvants and/or excipients.

An embodiment of the present disclosure includes a pharmaceutical composition or combination comprising of an ingredient selected from a first group of ingredients consisting of a lithium salt of eicosapentaenoic acid, a lithium salt of docosahexaenoic acid and one or more ingredient selected from a second group of ingredients consisting of aspirin, 2-hydroxypropyl-β-cyclodextrin, beta-hydroxy-beta-methylbutyrate, Caffeine, Cannabidiol.

An embodiment of the present disclosure includes a pharmaceutical composition or combination comprising of a lithium salt of aspirin and one or more ingredient selected from the group consisting of eicosapentaenoic acid, docosahexaenoic acid, herring roe oil, krill oil, linoleic acid, 2-hydroxypropyl-β-cyclodextrin, beta-hydroxy-beta-methylbutyrate, Caffeine, Cannabidiol.

An embodiment of the present disclosure includes a pharmaceutical composition or combination comprising of about 100 mg to about 10000 mg of stearidonic acid further comprising of phosphatidylinositol in amounts of about 0.8- about 50% of the weight of the total phospholipid composition further comprising of about 82 mg to about 800 mg or about 82 mg to about 200 mg of aspirin as active principles in association with one or more pharmaceutically acceptable adjuvants and/or excipients preferably 2-hydroxypropyl-β-cyclodextrin.

An embodiment of the present disclosure includes a pharmaceutical composition or combination comprising of about 100 mg to about 10000 mg stearidonic acid further comprising of about 82 mg to about 800 mg or about 82 mg to about 200 mg of aspirin further comprising of an effective amount of a COX2 inhibitor as active principles in association with one or more pharmaceutically acceptable adjuvants and/or excipients preferably 2-hydroxypropyl-3-cyclodextrin.

An embodiment of the present disclosure includes a pharmaceutical composition or combination comprising of about 100 mg to about 10000 mg of stearidonic acid further comprising of about 82 mg to about 800 mg or about 82 mg to about 200 mg of aspirin further comprising of about 125-μg or about 250-μg diosgenin as active principles in association with one or more pharmaceutically acceptable adjuvants and/or excipients preferably 2-hydroxypropyl-β-cyclodextrin.

An embodiment of the present disclosure includes a pharmaceutical composition or combination comprising of about 100 mg to about 10000 mg of eicosapentaenoic acid further comprising of about 82 mg to about 800 mg or about 82 mg to about 200 mg of aspirin further comprising of about 125-pg or about 250-pg diosgenin as active principles in association with one or more pharmaceutically acceptable adjuvants and/or excipients preferably 2-hydroxypropyl-β-cyclodextrin.

An embodiment of the present disclosure includes a pharmaceutical composition or combination comprising of about 100 mg to about 10000 mg of docosahexaenoic acid further comprising of about 82 mg to about 800 mg or about 82 mg to about 200 mg of aspirin further comprising of about 125-μg or about 250-μg diosgenin as active principles in association with one or more pharmaceutically acceptable adjuvants and/or excipients preferably 2-hydroxypropyl-β-cyclodextrin.

An embodiment of the present disclosure includes method of using of a pharmaceutical composition or combination embodiment of the present disclosure for treatment of diseases in a mammal selected from the group consisting of psoriasis, arthritic psoriasis, pediatric psoriasis, plaques, acne, atopic dermatitis, Danon Disease, depressive disorders, dyspraxia, multiple sclerosis, bone loss, colitis, inflammatory bowel disease, Crohn's Disease, ulcer, rheumatoid arthritis, cardiovascular disease (e.g., atherosclerosis, cardiomyopathy, congestive heart failure, endocarditis), renal disease, liver diseases (e.g., nonalcoholic fatty liver disease, nonalcoholic steatohepatitis, and primary biliary cholangitis), lung diseases (e.g., interstitial lung disease, idiopathic pulmonary fibrosis, hypersensitivity pneumonitis, sarcoidosis, and asbestosis), aging associated diseases (e.g., atherosclerosis, arthritis, cataracts, osteoporosis, type 2 diabetes, hypertension, Alzheimer's disease, hearing loss, maculopathy, osteoarthritis, Parkinson's disease, periodontitis, rheumatoid arthritis, and sarcopenia), post-traumatic stress disorder, autism spectrum disorders, stroke, Rett syndrome, Parkinson's disease, Alzheimer' s disease, diabetes, obesity, infertility, traumatic brain injury, skin disorders and various diseases caused by the hyperproliferation of cells such as cancers (e.g., colorectal cancer, osteomyelitis, Hodgkin Disease, Bladder cancer, prostate cancer, ovarian cancer, gall bladder cancer, pancreatic cancer, esophageal cancer, liver cancer, mesothelioma and lung cancer).

An embodiment of the present disclosure includes method of using of a pharmaceutical composition or combination embodiment of the present disclosure for reducing pain and inflammation.

An embodiment of the present disclosure includes method of using of a pharmaceutical composition or combination embodiment of the present disclosure for reducing gene expression or systemic levels of proteins selected from the group consisting of SQSTM1, COX 2, VEGF2, IL23, II17, IL1, IL6, TNF alpha, PON1.

An embodiment of the present disclosure includes method of using of a pharmaceutical composition or combination embodiment of the present disclosure for treatment of a disease associated with increased levels of SQSTM1 and COX2.

An embodiment of the present disclosure includes a method of treating a disease or the symptoms thereof in a mammal, the disease selected from the group consisting of psoriasis, arthritic psoriasis, pediatric psoriasis, plaques, acne, atopic dermatitis, Danon Disease, depressive disorders, dyspraxia, multiple sclerosis, bone loss, colitis, inflammatory bowel disease, Crohn's Disease, ulcer, rheumatoid arthritis, cardiovascular disease (e.g., atherosclerosis, cardiomyopathy, congestive heart failure, endocarditis), renal disease, liver diseases (e.g., nonalcoholic fatty liver disease, nonalcoholic steatohepatitis, and primary biliary cholangitis), lung diseases (e.g., interstitial lung disease, idiopathic pulmonary fibrosis, hypersensitivity pneumonitis, sarcoidosis, and asbestosis), aging associated diseases (e.g., atherosclerosis, arthritis, cataracts, osteoporosis, type 2 diabetes, hypertension, Alzheimer's disease, hearing loss, maculopathy, osteoarthritis, Parkinson's disease, periodontitis, rheumatoid arthritis, and sarcopenia), post-traumatic stress disorder, autism spectrum disorders, stroke, Rett syndrome, Parkinson's disease, Alzheimer's disease, diabetes, obesity, infertility, traumatic brain injury, skin disorders and diseases caused by the hyperproliferation of cells including cancer (e.g., colorectal cancer, osteomyelitis, Hodgkin Disease, Bladder cancer, prostate cancer, ovarian cancer, gall bladder cancer, pancreatic cancer, esophageal cancer, liver cancer, mesothelioma and lung cancer), the method comprising administering to said mammal in need of such treatment an effective amount of at least one of a first group consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil; an effective amount of at least one of a second group consisting of phosphatidylinositol, inositol, and aspirin; and an effective amount of at least one of a third group consisting of 2-hydroxypropyl-β-cyclodextrin, beta-hydroxy-beta-methylbutyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus.

An embodiment of the present disclosure includes a method of reducing pain and inflammation or the symptoms thereof in a mammal, the method comprising administering to said mammal in need of such treatment an effective amount of at least one of a first group consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil; an effective amount of at least one of a second group consisting of phosphatidylinositol, inositol, and aspirin; and an effective amount of at least one of a third group consisting of 2-hydroxypropyl-β-cyclodextrin, beta-hydroxy-beta-methylbutyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus.

An embodiment of the present disclosure includes a method of reducing gene expression or systemic levels of proteins selected from the group consisting of SQSTM1, COX 2, VEGF2, IL23, 1117, IL1, IL6, TNF alpha, and PON1 in a mammal, the method comprising administering to said mammal in need of such treatment an effective amount of at least one of a first group consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil; an effective amount of at least one of a second group consisting of phosphatidylinositol, inositol, and aspirin; and an effective amount of at least one of a third group consisting of 2-hydroxypropyl-β-cyclodextrin, beta-hydroxy-beta-methylbutyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus.

An embodiment of the present disclosure includes a method of treating a disease associated with increased levels of SQSTM1 or COX2 in a mammal, the method comprising administering to said mammal in need of such treatment an effective amount of at least one of a first group consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil; an effective amount of at least one of a second group consisting of phosphatidylinositol, inositol, and aspirin; and an effective amount of at least one of a third group consisting of 2-hydroxypropyl-β-cyclodextrin, beta-hydroxy-beta-methylbutyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus.

An embodiment of the present disclosure includes a method of treating a disease or the symptoms thereof in a mammal, the disease selected from the group consisting of psoriasis, arthritic psoriasis, pediatric psoriasis, plaques, acne, and atopic dermatitis, the method comprising administering to said mammal in need of such treatment an effective amount of at least one of a first group consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil; an effective amount of at least one of a second group consisting of phosphatidylinositol, inositol, and aspirin; and an effective amount of at least one of a third group consisting of 2-hydroxypropyl-β-cyclodextrin, beta-hydroxy-beta-methylbutyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus.

### EXAMPLE

Evaluation of analgesic and anti-inflammatory effects of compositions of claim 1 (See Hunskaar S and Hole K. (1987) Pain 30(1):103-14.)

In the Formalin Test a 5% solution of formaldehyde is injected subcutaneously to rat paw which produces a biphasic pain response over a test period of 60 minutes: an early phase lasting the first 5 min and a late phase lasting from 20 to 30 min after the injection of formalin. The early phase is due to a direct effect of compositions on nociceptors and the late phase seems to be an inflammatory response with inflammatory pain that can be inhibited by anti-inflammatory drugs. For determining effects of compositions on these response phases two measurements were taken with three minutes interval for determining the number of flinches and licks per minute.

The treatments administered consists of (Human equivalent dose)

1. Control

2. Anacin 500 (consisting of 500 mg Aspirin and 65 mg Caffeine)

3. Anacin 800 (consisting of 800 mg Aspirin and 65 mg Caffeine)

4. KPAC composition consisting of 800 mg Krill oil, 500 mg Aspirin, 65 mg Caffeine 65 mg and 800 mg of the autophagy activator Pterostilbene

The above ingredients were suspended in 2.5 ml saline solution to feed by gavage one hour before injection of formalin and pain response was measured for 45 minutes. The data were collected in duplicate and average was taken.

TABLE 2
Results:

| | Control | | | | Anacin-500 | | | |
|---|---|---|---|---|---|---|---|---|
| | 1 | | 2 | | 1 | | 2 | |
| Time in Minutes | Flinches | Licks | Flinches | Licks | Flinches | Licks | Flinches | Licks |
| 0-3 | 8 | 14 | 4 | 5 | 8 | 18 | 8 | 13 |
| 3-6 | 8 | 1 | 7 | 6 | 2 | 2 | 4 | 4 |
| 6-10 | 3 | 0 | 5 | 2 | 3 | 0 | 2 | 3 |
| 11-13 | 3 | 4 | 5 | 3 | 3 | 3 | 7 | 3 |
| 15-18 | 8 | 1 | 5 | 8 | 2 | 4 | 2 | 6 |
| 21-24 | 10 | 7 | 14 | 11 | 1 | 11 | 2 | 10 |
| 25-28 | 17 | 7 | 10 | 12 | 7 | 14 | 5 | 8 |
| 31-33 | 12 | 13 | 11 | 9 | 1 | 11 | 1 | 7 |
| 35-38 | 14 | 14 | 19 | 17 | 10 | 12 | 4 | 8 |
| 41-43 | 15 | 1 | 14 | 4 | 5 | 0 | 4 | 3 |

| | Anacin-800 | | | | KPAC | | | |
|---|---|---|---|---|---|---|---|---|
| | 1 | | 2 | | 1 | | 2 | |
| Time in Minutes | Flinches | Licks | Flinches | Licks | Flinches | Licks | Flinches | Licks |
| 0-3 | 2 | 0 | 0 | 3 | 0 | 5 | 0 | 3 |
| 3-6 | 0 | 0 | 0 | 0 | 0 | 2 | 0 | 3 |
| 6-10 | 2 | 1 | 0 | 0 | 0 | 1 | 0 | 0 |
| 11-13 | 0 | 0 | 0 | 0 | 0 | 1 | 0 | 0 |
| 15-18 | 0 | 0 | 0 | 4 | 2 | 1 | 0 | 0 |
| 21-24 | 0 | 0 | 1 | 3 | 2 | 4 | 3 | 3 |
| 25-28 | 0 | 0 | 0 | 4 | 0 | 5 | 0 | 6 |
| 31-33 | 1 | 1 | 0 | 1 | 1 | 4 | 3 | 6 |
| 35-38 | 0 | 0 | 0 | 1 | 1 | 4 | 0 | 4 |
| 41-43 | 0 | 0 | 0 | 0 | 0 | 2 | 0 | 4 |

Results: KPAC inhibits nociceptive and anti-inflammatory pain responses superior to that of a 500 mg doses of Anacin and comparable to that of a high dose Anacin. This observation indicates that compositions of claim 1 have utility for treatment of acute pain and inflammatory conditions.

TABLE 3
Summary of results of experiments determining percent inhibition of substance combinations in
formalin induced pain responses lasting from 20 to 30 min after the injection of formalin.

| | | | | | | | Krill oil | Krill oil |
|---|---|---|---|---|---|---|---|---|
| | | | | | | | 400 mg + | 400 mg + |
| | | | | | Krill oil | | Aspirin | CBD |
| Human | | | | | 400 mg + | | 100 mg + | 12.5 mg + |
| equivalent | | | | | Inositol | Cannabidiol | CBD | Inositol |
| Doses in mg | Paracetamol | Aspirin | Krill oil | Inositol | 250 mg | (CBD) | 12.5 mg | 250 mg |
| 3 | | | | | | 8 | | |
| 6.25 | | | | | | 40 | | |
| 12.5 | | | | | | 40 | 90 | 90 |
| 25 | | | | | | 90 | | |
| 31.5 | | | | 12 | | | | |
| 50 | | | | | | 90 | | |
| 60.2 | | | | 30 | | | | |
| 100 | | 30 | 10 | | | | | |
| 125 | | | | 34 | | | | |
| 200 | | | 20 | | | | | |
| 250 | | | | 40 | | | | |
| 400 | | | 44 | | 76 | | | |
| 500 | 50 | | | 85 | | | | |
| 800 | | | 60 | | | | | |
| 1000 | 70 | | | | | | | |
| 1600 | | | 85 | | | | | |

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1. A pharmaceutical composition comprising at least one of a first group consisting of herring roe oil, krill oil, fish oils, eicosapentaenoic acid, docosahexaenoic acid, docosapentaenoic acid, stearidonic acid, lithium and zinc salts of docosahexaenoic acid, lithium and zinc salts of docosapentaenoic acid, lithium and zinc salts of eicosapentaenoic acid, lithium and zinc salts of stearidonic acid, linoleic acids, and a vegetable oil containing one or more of said linoleic acids in amounts of at least 8 percent of the lipid composition of said vegetable oil; at least one of a second group consisting of phosphatidylinositol, inositol, and aspirin; and at least one of a third group consisting of 2-hydroxypropyl-β-cyclodextrin, beta-hydroxy-beta-methyl butyrate; 6-shogaol, acacetin, acetyl-11-keto-boswellic acid, alexidine, all-trans-retinoic acid, ambroxol, amiodarone, AP23841, ascomycin, astragalus sinicus extracts, atorvastatin, simvastatin, lovastatin, fluvastatin, pravastatin, cerivistatin, monascus purpureus, rosuvastatin, AZD08055, berberine, berberis aristata extracts, bilobalide, boswellia serrata extracts, caffeine, cannabinoids, cannabidiol, cepharanthine, curcumin, dauricine, deferolimus, diclofenac, diosgenin, epigallocatechin gallate, estrogen, everolimus, EX2044, EX3855, EX7518, fasudil, gallic acid, ganoderma lucidum extract, genkwanin, genistein, glucosamine, hernandezine, HU-308, hydroxytyrosol, INK-128, isorhamnetin, KU-0063794, lanthionine ketimine and its brain penetrable ethyl ester, LY303511, mahonia aquifolium extracts, melatonin, metformin, monascus purpureus, NCGC758, niclosamide, NV-128, oleuropein, oregon grape root extracts, OSI-027, PI 3-kinase inhibitors, pioglitazone, perhexiline, plerixafor, pterostilbene, the root of polygonum cuspidatum containing resveratrol, perifosine, pyrroloquinoline quinone, quercetin, quercitrin, resveratrol, ripasudil, rosglitazone, sirolimus, spermidine, telmisartan, temsirolimus, thalidezine, the root of the curcuma longa containing curcumin, troglitazone, urolithin A, urolithin B, urolithin C, urolithin D, vitis vinifera extracts containing resveratrol, WYE-125132, xestospongin B, XL765, zinc acetate, zinc aspartate, zinc carbonate, zinc citrate, zinc gluconate, zinc oxide, zinc sulfate, and zotarolimus; and a pharmaceutically acceptable carrier.

2. The pharmaceutical composition according to claim 1, wherein the at least one of the first group, the at least one of the second group and the at least one of the third group include an effective amount thereof.

3. The pharmaceutical composition according to claim 2, wherein the at least one of the second group is aspirin or inositol.

4. The pharmaceutical composition according to claim 2, wherein the at least one of the second group is inositol.

5. The pharmaceutical composition according to claim 4, wherein the at least one of the first group is eicosapentaenoic acid.

6. The pharmaceutical composition according to claim 4, wherein the at least one of the first group is docosahexaenoic acid.

7. The pharmaceutical composition according to claim 4, wherein the at least one of the first group is krill oil.

8. The pharmaceutical composition according to claim 4, wherein the at least one of the first group is herring roe oil.

9. The pharmaceutical composition according to claim 4, wherein the at least one of the third group is selected from the group consisting of all-trans-retinoic acid, atorvastatin, berberine, beta-hydroxy-beta-methylbutyrate, caffeine, cannabidiol, curcumin, glucosamine, melatonin, metformin, niclosamide, perhexiline, perifosine, pioglitazone, pterostilbene, pyrroloquinoline quinone, quercetin, resveratrol, ripasudil, rosglitazone, tretinoin, troglitazone, zinc acetate, zinc aspartate, zinc carbonate, zinc citrate, zinc gluconate, zinc oxide, and zinc sulfate.

10. The pharmaceutical composition according to claim 9, wherein the at least one of the third group is metformin .

11. The pharmaceutical composition according to claim 9, wherein the at least one of the third group is melatonin.

12. The pharmaceutical composition according to claim 9, wherein the at least one of the third group is niclosamide.

13. The pharmaceutical composition according to claim 9, wherein the at least one of the third group is pterostilbene.

14. The pharmaceutical composition according to claim 4 wherein the at least one of the third group of ingredients is cannabidiol.

15. A pharmaceutical composition comprising: herring roe oil in an amount of about 100 mg to about 10000 mg; inositol in an amount of about 100 mg to about 10000 mg;
about 0 mg to about 200 mg of aspirin, pterostilbene in an amount of about 100 mg to about 1000 mg and a pharmaceutically acceptable carrier.

16. A pharmaceutical composition comprising: docosahexaenoic acid in an amount of about 100 mg to about 10000 mg; inositol in an amount of about 100 mg to about 10000 mg; cannabidiol in an amount of about 5 mg to about 250 mg and a pharmaceutically acceptable carrier.

17. A pharmaceutical composition comprising: herring roe oil in an amount of about 100 mg to about 10000 mg; inositol in an amount of about 100 mg to about 10000 mg;
cannabidiol in an amount of about 2 mg to about 100 mg; caffeine in an amount of about 0 mg to about 100 mg and a pharmaceutically acceptable carrier.

18. A pharmaceutical composition comprising: eicosapentaenoic acid in an amount of about 100 mg to about 10000 mg; inositol in an amount of about 100 mg to about 10000 mg; pterostilbene in an amount of about 5 mg to about 500 mg; and a pharmaceutically acceptable carrier.

19. A pharmaceutical composition comprising: eicosapentaenoic acid in an amount of about 100 mg to about 10000 mg; inositol in an amount of about 100 mg to about 10000 mg; metformin in an amount of about 100 mg to about 2500 mg and a pharmaceutically acceptable carrier.

20. A pharmaceutical composition comprising: eicosapentaenoic acid in an amount of about 100 mg to about 10000 mg; inositol in an amount of about 100 mg to about 10000 mg; pterostilbene in an amount of about 5 mg to about 500 mg; niclosamide in an amount of 500 mg to 2000 mg and a pharmaceutically acceptable carrier.

21. A pharmaceutical composition comprising: krill oil in an amount of about 100 mg to about 10000 mg; inositol in an amount of about 100 mg to about 10000 mg; pterostilbene in an amount of about 5 mg to about 500 mg; caffeine in an amount of about 0 mg to about 100 mg and a pharmaceutically acceptable carrier.

22. A method of treating a disease or the symptoms thereof in a mammal, the disease selected from the group consisting of psoriasis, arthritic psoriasis, pediatric psoriasis, plaques, acne, atopic dermatitis, Danon Disease, depressive disorders, dyspraxia, multiple sclerosis, bone loss, colitis, inflammatory bowel disease, Crohn's Disease, ulcer, rheumatoid arthritis, cardiovascular disease including atherosclerosis, cardiomyopathy, congestive heart failure or endocarditis, liver diseases including nonalcoholic fatty liver disease, nonalcoholic steatohepatitis, or primary biliary cholangitis, lung diseases including interstitial lung disease, idiopathic pulmonary fibrosis, hypersensitivity pneumonitis, sarcoidosis or asbestosis, aging associated diseases including atherosclerosis, arthritis, cataracts, osteoporosis, type 2 diabetes, hypertension, Alzheimer's disease, hearing loss, maculopathy, osteoarthritis, Parkinson's disease, periodontitis, rheumatoid arthritis or sarcopenia, post-traumatic stress disorder, autism spectrum disorders, stroke, Rett syndrome, obesity, infertility, traumatic brain injury and diseases caused by the hyperproliferation of cells including colorectal cancer, osteomyelitis, Hodgkin Disease, Bladder cancer, prostate cancer, ovarian cancer, gall bladder cancer, pancreatic cancer, esophageal cancer, liver cancer, mesothelioma, and lung cancer, the method comprising administering to said mammal in need of such treatment an effective amount of a pharmaceutical composition of claim 1.

23. A method of reducing pain and inflammation or the symptoms thereof in a mammal, the method comprising administering to said mammal in need of such treatment an effective amount of a pharmaceutical composition of claim 1.

24. A method of treating a disease associated with increased levels of SQSTM1 or COX2 in a mammal, the method comprising administering to said mammal in need of such treatment an effective amount of a pharmaceutical composition of claim 1.
